SEQUENCE LISTING (5 sequences)

SEQ ID NO: 1 - protein, 815 aa, Homo sapiens
MAEPLKEEDGEDGSAEPPAREGRTRPHRCLCSAKNLALLKARSFDVTFDVGDEYEIIETIGNGAYGVVSSARRRLTGQQVAIKKIPNAFDVVTNAKRTLRELKILKHFKHDNIIAIKDILRPTVPYGEFKSVYVVLDLMESDLHQIIHSSQPLTLEHVRYFLYQLLRGLKYMHSAQVIHRDLKPSNLLVNENCELKIGDFGMARGLCTSPAEHQYFMTEYVATRWYRAPELMLSLHEYTQAIDLWSVGCIFGEMLARRQLFPGKNYVHQLQLIMMVLGTPSPAVIQAVGAERVRAYIQSLPPRQPVPWETVYPGADRQALSLLGRMLRFEPSARISAAAALRHPFLAKYHDPDDEPDCAPPFDFAFDREALTRERIKEAIVAEIEDFHARREGIRQQIRFQPSLQPVASEPGCPDVEMPSPWAPSGDCAMESPPPAPPPCPGPAPDTIDLTLQPPPPVSEPAPPKKDGAISDNTKAALKAALLKSLRSRLRDGPSAPLEAPEPRKPVTAQERQREREEKRRRRQERAKEREKRRQERERKERGAGASGGPSTDPLAGLVLSDNDRSLLERWTRMARPAAPALTSVPAPAPAPTPTPTPVQPTSPPPGPLAQPTGPQPQSAGSTSGPVPQPACPPPGPAPHPTGPPGPIPVPAPPQIATSTSLLAAQSLVPPPGLPGSSTPGVLPYFPPGLPPPDAGGAPQSSMSESPDVNLVTQQLSKSQVEDPLPPVFSGTPKGSGAGYGVGFDLEEFLNQSFDMGVADGPQDGQADSASLSASLLADWLEGHGMNPADIESLQREIQMDSPMLLADLPDLQDP

SEQ ID NO: 2 - DNA, 2828 bp, Homo sapiens
gaattccggagacccccgcgctggggacgggaggccggcgagcctcgggacctctgaaagccttgaggaggcccggggacaccatggccgagcctctgaaggaggaagacggcgaggacggctctgcggagcccccggcccgtgaaggtcgaacccgcccacaccgctgcctctgtagcgccaagaacctggccctgcttaaagcccgctccttcgatgtgacctttgacgtgggcgacgagtacgagatcatcgagaccataggcaacggggcctatggagtggtgtcctccgcccgccgccgcctcaccggccagcaggtggccatcaagaagatccctaatgctttcgatgtggtgaccaatgccaagcggaccctcagggagctgaagatcctcaagcactttaaacacgacaacatcatcgccatcaaggacatcctgaggcccaccgtgccctatggcgaattcaaatctgtctacgtggtcctggacctgatggaaagcgacctgcaccagatcatccactcctcacagcccctcacactggaacacgtgcgctacttcctgtaccaactgctgcggggcctgaagtacatgcactcggctcaggtcatccaccgtgacctgaagccctccaacctattggtgaatgagaactgtgagctcaagattggtgactttggtatggctcgtggcctgtgcacctcgcccgctgaacatcagtacttcatgactgagtatgtggccacgcgctggtaccgtgcgcccgagctcatgctctctttgcatgagtatacacaggctattgacctctggtctgtgggctgcatctttggtgagatgctggcccggcgccagctcttcccaggcaaaaactatgtacaccagctacagctcatcatgatggtgctgggtaccccatcaccagccgtgattcaggctgtgggggctgagagggtgcgggcctatatccagagcttgccaccacgccagcctgtgccctgggagacagtgtacccaggtgccgaccgccaggccctatcactgctgggtcgcatgctgcgttttgagcccagcgctcgcatctcagcagctgctgcccttcgccaccctttcctggccaagtaccatgatcctgatgatgagcctgactgtgccccgccctttgactttgcctttgaccgcgaagccctcactcgggagcgcattaaggaggccattgtggctgaaattgaggacttccatgcaaggcgtgagggcatccgccaacagatccgcttccagccttctctacagcctgtggctagtgagcctggctgtccagatgttgaaatgcccagtccctgggctcccagtggggactgtgccatggagtctccaccaccagccccgccaccatgccccggccctgcacctgacaccattgatctgaccctgcagccacctccaccagtcagtgagcctgccccaccaaagaaagatggtgccatctcagacaatactaaggctgcccttaaagctgccctgctcaagtctttgaggagccggctcagagatggccccagcgcacccctggaggctcctgagcctcggaagccggtgacagcccaggagcgccagcgggagcgggaggagaagcggcggaggcggcaagaacgagccaaggagcgggagaaacggcggcaggagcgggagcgaaaggaacggggggctggggcctctgggggcccctccactgaccccttggctggactagtgctcagtgacaatgacagaagcctgttggaacgctggactcgaatggcccggcccgcagccccagccctcacctctgtgccggcccctgccccagcgccaacgccaaccccaaccccagtccaacctaccagtcctcctcctggccctctagcccagcccactggcccgcaaccacaatctgcgggctctacctctggccctgtaccccagcctgcctgcccaccccctggccctgcaccccaccccactggccctcctgggcccatccctgtccccgcgccaccccagattgccacctccaccagcctcctggctgcccagtcacttgtgccaccccctgggctgcctggctccagcaccccaggagttttgccttacttcccacctggcctgccgcccccagacgccgggggagcccctcagtcttccatgtcagagtcacctgatgtcaaccttgtgacccagcagctatctaagtcacaggtggaggaccccctgccccctgtgttctcaggcacaccaaagggcagtggggctggctacggtgttggctttgacctggaggaattcttaaaccagtctttcgacatgggcgtggctgatgggccacaggatggccaggcagattcagcctctctctcagcctccctgcttgctgactggctcgaaggccatggcatgaaccctgccgatattgagtccctgcagcgtgagatccagatggactccccaatgctgctggctgacctgcctgacctccaggacccctgaggcccccagcctgtgccttgctgccacagtagacctagttccaggatccatgggagcattctcaaaggctttagccctggacccagcaggtgaggctcggcttggattattctgcaggttcatctcagacccacctttcagccttaagcagccacctgagccaccaccgagccatggcaggatcgggagaccccaactccccctgaacaatccttttcagtattatatttttattattattatgttattattacactgtcttttgccatcaaaatgaggcctgtgaaatacaaggtt

SEQ ID NO: 3 - DNA, 53 bp, Artificial
  shERK5 (1)
agctgccctgctcaagtctttcaagagaagacttgagcagggcagcttttttt

SEQ ID NO: 4 - DNA, 53 bp, Artificial
  shERK5 (2)
ccagtccaacctaccagtcctctcgagaggactggtaggttggactggttttt

SEQ ID NO: 5 - DNA, 53 bp, Artificial
  shERK5 (3)
gccaagtaccatgatcctgatctcgagatcaggatcatggtacttggcttttt

(19) United States
(12) Patent Application Publication
(10) Pub. No.: US 2011/0150936 A1
(43) Pub. Date: Jun. 23, 2011

(54) ANTI-TUMORAL CELLS

(76) Inventors: Martin Villalba Gonzales, Montpellier (FR); Robert Hipskind, Saint Gely Du Fesc (FR); Johan Garaude, Montpellier (FR); Seyma Cherni, Montpellier (FR); Geoffroy De Bettignies, Montarnaud (FR); Chantal Jacquet, Lunel (FR)

(21) Appl. No.: 12/994,123

(22) PCT Filed: May 22, 2009
(86) PCT No.: PCT/IB2009/005923

§ 371 Date: Mar. 3, 2011

(30) Foreign Application Priority Data
May 23, 2008 (FR) 08/02809

(51) Int. Cl. A61K 35/12; C12N 5/09; C12N 5/10; C12N 5/0783; A61K 39/00; A61K 31/713; A61K 31/7088; A61P 35/00; A61P 37/04

(52) U.S. Cl. 424/277.1; 435/325; 435/375; 435/366; 424/93.7; 514/44 A; 514/44 R

(57) ABSTRACT

The present invention relates to the field of preventive or therapeutic anti-tumoral vaccine. More specifically, the present invention relates to live animal tumour cells having a negative MHC-I phenotype, to methods for the production of such MHC-I negative cells, and to their therapeutic use as anti-tumoral agents, particularly via their capacity to activate natural killer (NK) cells. The invention further relates to methods for modulating the level of expression of MHC-I molecules on animal tumour cells, for example by use of specific culture conditions, and/or by use of exogenous agents which directly or indirectly affect levels of MHC-I expression. The invention also concerns activated NK cells and their therapeutic use as anti-tumoral agents.

The present invention relates to the field of preventive or therapeutic anti-tumoral vaccine. More specifically, the present invention relates to live animal tumour cells having a negative MHC-I phenotype, to methods for the production of such MHC-I negative cells, and to their therapeutic use as anti-tumoral agents, particularly via their capacity to activate natural killer (NK) cells. The invention further relates to methods for modulating the level of expression of MHC-I molecules on animal tumour cells, for example by use of specific culture conditions, and/or by use of exogenous agents which directly or indirectly affect levels of MHC-I expression. The invention also concerns activated NK cells and their therapeutic use as anti-tumoral agents.

Recent observations have alleviated doubts regarding the existence of an effective immune response which is capable of curing cancer at a clinical level. However, tumoral antigens or tumor-associated antigens (TAA) have proved to be weak immunogens at best. Vaccines based on tumor cells constitute an interesting alternative to immunization with peptides or cDNA. Currently, they generally comprise apoptotic cells or cell extracts. In fact, some of the first clinical trials used irradiated autologous tumor cells to boost the anti-tumoral immune response (1). However, apoptotic cells or cell extracts have not proved to be strong immunogens and lead to deficient activation of the immune system against tumor cells. Living cells should be better immunogens than dying cells, but such tumor cells have to be eliminated rapidly and effectively by the host immune system which must also be highly activated, leading to tumoral immunization. Efforts are now principally directed towards the development by genetic engineering of effector cells, for example dendritic cells (DC). Another possibility, albeit not explored in detail, consists of altering the tumor cells to render them highly immunogenic. One example could be to increase the number of tumor cells undergoing apoptosis in order to induce a satisfactory immune response. The kinase ERK5 (“extracellular-regulated kinase 5”) or MAPK7 (“mitogen-activated protein kinase 7”) (2, 3), present in normal and leukemic T cells (4), shares the TEY (Thr-Glu-Tyr) activation motif with other ERK kinases. Its other structural features are unique, such as the large regulatory C terminus which controls its nucleo-cytoplasmic shuttling (5). ERK5 mediates survival and proliferative signaling dependent on ErbB (6), Ras (7), serum (8), IGF-II (9), Bcr-Abl (10) and IL-6 (11). Various groups have described the involvement of the ERK5 cascade in tumor cells. As an example, it has been shown that ERK5 plays a significant role in the proliferation of breast cancer cells (6, 8). A link has also been established between over-expression of the MEK5 kinase which activates ERK5 and the development of bony metastases leading to a poor prognosis in human prostate cancer (12). ERK5 is also necessary for chemoresistance in breast cancer cells (13) and it mediates cell survival in lung cancer cells (9). ERK5 is also expressed in myeloma cells, where its inhibition blocks proliferation and facilitates aptoptosis induced by dexamethasone (11). ERK5 expression is also essential for the survival of leukemic cells expressing Bcr/Abl (10). Furthermore, all Hodgkin lymphoma cells express a constitutively active ERK5 pathway (14). Finally, expression of miRNAs (or micro RNA or miR)-143 and -145, which have been shown to have reduced levels of expression in colon cancer cells and in different tumor cell lines, is also reduced in the majority of malignant B lymphocytes, including chronic lymphoid leukemia cells (CLL), type B lymphomas, B lymphocyte lines transformed with Epstein-Barr virus (EBV) and Burkitt lymphoma cell lines. All of the samples tested from 13 patients with a CLL and eight out of nine patients with a type B lymphoma showed very low levels of expression of miR-143 and -145. The levels of expression of miR-143 and -145 are also low in human cell lines of Burkitt's lymphoma and are inversely correlated with the proliferation of cells from B lymphocyte cell lines transformed with Epstein-Barr virus (EBV). Further, the introduction of precursor or mature miR-143 into Raji cells leads to a significant dose-dependent inhibition of growth, and ERK5 has been identified as being one of the targets of miR-143, confirming the results obtained with human colon cancer cells (35).

In vivo, invalidation of ERK5 or MEK5 genes affects both embryonic angiogenesis and cardiac development and leads to embryonic lethality by day 11.5 (15-17). The importance of the function of ERK5 in endothelial cells is confirmed by targeted deletion of ERK5 in adult mice, which perturbs vascular integrity and leads to death through endothelial failure (18). ERK5 is also required for tumor-associated neovascularization. In ERK5 flox/flox mice carrying the Mx1-Cre transgene, deletion of the host ERK5 gene reduces the volume of tumor xenografts and leads to a significant decrease in vascular density (19). Thus, ERK5 plays a central role in several processes linked to tumorigenesis: transduction of intracellular oncogenic signals, tumor-associated angiogenesis and, in some cases, invasion/metastasis. Interestingly, ERK5−/− MEF cells undergo apoptosis under basal conditions and ERK5+/− MEFs are also sensitized to sorbitol-induced apoptosis, suggesting that a small reduction in ERK5 levels has significant effects (20); this has also been observed by the inventors (21).

These observations suggest that ERK5 plays an important role in oncogenesis, the mechanism of which remains poorly characterized. Recent results obtained by the inventors (21) show that ERK5 mediates activation of the anti-apoptotic transcription factor NF-κB in human and murine leukemic T cells. ERK5 knockdown by “shERK5”, a small hairpin RNA (shERK5), reduces cell viability and sensitizes cells to death receptor-induced apoptosis. The inventors have also shown in their previous work that cells from the EL4 T lymphocyte line derived from a murine lymphocyte expressing shERK5 (EL4-shERK5 cells), proliferate in vitro but fails to induce subcutaneous tumors in vivo in mice. These data suggest that ERK5 is essential for survival of human and murine leukemic T lymphocytes and thus represent a promising target for therapeutic intervention in this type of malignancy.

The immune system is a complex integrated system which uses cells produced in the bone marrow, in particular B, T and NK lymphocytes, dendritic cells, Langherhans cells, monocytes, macrophages, neutrophils, mastocytes, basophils and eosinophils. A distinction can be made between the “innate” or “natural” immune response which has the major characteristic of being immediate and not antigen-specific, and the “adaptive” or “acquired” response, which has the major characteristic of being retarded and antigen-specific. The adaptive response also has the characteristic, in contrast to the innate response, of triggering a reaction against a second attack by the same antigen (secondary response) which is more effective and more rapid than the primary response.

Natural killer (NK) cells, which play a critical role in innate anti-tumor immunity, spontaneously kill cells which behave abnormally, for example tumor cells, while sparing healthy cells. In particular, NK cells kill tumor cells that have evaded the control of cytotoxic T lymphocytes (CTL) by downregulating the level of expression of the major histocompatibility complex class I (MHC-I). The activation or inhibition of NK cells is finely adjusted by integrating signals deriving either from inhibiting receptors or from activating receptors. All of these signals control the lytic activity of NK cells which release perforins and granzymes or express death receptor ligands, causing the death of target cells by apoptosis. Inhibitor or “KIR” receptors are specific to MHC class I molecules. In vitro, NK cells can be recruited locally by inoculation of tumor cells, preferably those lacking appropriate MHC-I expression (22). Their versatility makes them attractive targets to be exploited in clinical approaches to cancer (23, 24). NK cells may be of particular benefit in blood-borne cancers, such as leukemias and lymphomas, due to the abundance of NK cells in the peripheral blood and spleen. Further, new data suggest that NK cell-based immunonotherapy may be used in combination with stem cell transplantation (SCT). Pilot clinical studies have shown that adoptive transfer of donor-derived NK cells consolidates engraftment in patients with acute myeloid leukemia (“AML”) following haploidentical SCT (25, 26).

Most tumor cells change from aerobic glycolysis (respiration) to a more anaerobic-like glycolysis (fermentation) during the first steps of tumorigenesis and therefore, they perform less oxidative phosphorylation (OXPHOS). This change known as the Warburg effect ³⁷ involves specific gene expression ³⁸. At this step, tumor cells must also avoid the attack from the host immune system ³⁸, and the most extended mechanism is downregulation of plasma membrane expression of the major histocompatibility complex I (MHC-I; ⁴⁰). The inventors have shown here that both phenomena are linked: fermentative-growing tumor cells forced to perform respiration upregulate expression of MHC-I, and vice versa, tumor cells growing in OXPHOS media reduce MHC-I expression when changed to fermentative conditions. Respiration induces MHC-I expression at the transcriptional level because tumor cells performing respiration show significant higher mRNA levels of heavy and light chains of class I molecules. The expression of the MAPK ERK5, which activates promoters of MHC-I genes ⁴¹, is upregulated in OXPHOS conditions and accumulates in mitochondria. This process is essential for tumor cell survival and MHC-I expression under OXPHOS conditions. The results obtained by the inventors show that changes in tumor cell metabolism modulates ERK5 expression leading to modifications in MHC-I expression. The inventors also have shown that in tumor cells growing under OXPHOS conditions most ERK5 was isolated in the mitochondrial fraction and that shERK5 cells showed a significant increase in cell death when forced to perform respiration, thus suggesting a role of ERK5 in respiration. In summary, the change from respiration to fermentation offers tumor cells the possibility of downregulate their MHC-I and confers a selective advantage: escape of the immune response.

The inventors have now shown on the one hand the fact that under certain culture conditions, tumor cells modified in vitro or ex vivo to present a negative ERK5 phenotype also have a negative MHC-I phenotype, and on the other hand the fact that tumor cells presenting a negative MHC-I phenotype and preferably also a negative ERK5 phenotype, may be used as a live anti-tumoral vaccine. They have in fact shown that administering tumor cells presenting a negative ERK5 phenotype and a negative MHC-I phenotype to mice induces an innate immune response involving NK cells against not only the modified tumor cells but also against “endogenous” tumor cells with a positive MHC-I phenotype.

This can be explained by the fact that NK cells, once activated by the administered tumor cells, are capable of lysing positive MHC-I tumor cells because of activation of activating receptors such as CD94/NKG2C, NKG2D, KIR2DS. The antigens MIC-A, MIC-B (two MHC-I related molecules induced by stress and interacting directly with the activating receptor NKG2D) and hsp70 expressed by “endogenous” tumor cells (“wt” or “wild type”) will then be recognized by activated NK cells and be capable of triggering lysis of the tumor cells despite the fact that they have a positive MHC-I phenotype. Further, it is probable that presentation by dendritic cells of antigens derived from the lysis of cancer cells by NK cells also activates an adaptive immune response involving cytotoxic T lymphocytes (CTL).

Further, the inventors have demonstrated in the mouse that using tumor cells cumulating negative ERK5 phenotype and a negative MHC-I phenotypes has an advantage as regards the effectiveness of the anti-tumoral action on the use of cells having just a negative MHC-I phenotype (see Example 1, Table 1), suggesting that the anti-tumoral effect is not linked only to the negative MHC-I phenotype and that the negative ERK5 phenotype can produce a supplemental anti-tumoral effect. The results presented here show in particular that tumor cells modified in vitro or ex vivo to present a negative ERK5 phenotype have a high level of expression of Fas and a low level of expression of the anti-apoptotic protein c-FLIP (FIGS. 12, 16, 17). As a result, these cells are excellent targets for cells expressing FasL, such as NK cells and cytotoxic T lymphocytes. Further, the expression of c-FLIP is regulated by NF-κB for which it has been shown that activation is regulated by ERK5 (21).

Finally, the inventors have demonstrated that tumor cells combining negative ERK5 and negative MHC-I phenotypes are rapidly eliminated from the organism (in less than three days for the mouse). As a consequence, these cells are adapted to use in therapy.

More particularly, the inventors have focused on the promotion of the immune response which may be induced in the mouse by EL4 tumor cells modified to express the short RNA “shERK5” which, by dint of an interference mechanism, can reduce the level of expression of the ERK5 protein in the cell. They have shown that “attenuated” cancer cells modified to have a negative ERK5 phenotype and a negative MHC-I phenotype are capable of protecting mice from leukemia.

The inventors have also shown that EL4-shERK5 cells are eliminated three days following injection, in other words well before the time necessary for triggering an adaptive immune response. The inventors have assumed that the innate immune system, in particular NK cells, is involved in this response. The results obtained have confirmed this hypothesis, showing that NK cells effectively eliminate EL4-shERK5 cells in vivo principally because of the large reduction in the level of expression of the major histocompatibility complex class I (MHC-I) by these cells and their increased sensitivity to death receptors.

The inventors have hypothesized that such activation of the immune system could induce an immune response against tumors caused by wild type (“wt”) cells. This hypothesis has been confirmed by short term and long term experiments. In contrast to Yac-1 cells derived from a lymphoma induced by the M-MLV virus, EL4 cells are derived from a lymphoma induced in the C57BL mouse by 9,10-dimethyl-1,2-benzanthracene. The immune response induced by EL4-shERK5 cells protected the mice against leukemias induced by retroviruses. Further, EL4-shERK5 cells protected the mice more effectively than Yac-1 cells which also have a greatly diminished level of expression of MHC-I. Further, the EL4-shERK5 cells have a diminished activation of NF-κB, which renders them highly sensitive to apoptosis induced by death receptors (21). This could explain why these cells are better targets for NK cells than Yac-1 cells, and induce as effective an anti-tumoral immune response. In this context, NK cells play an essential role in the anti-tumoral immune response against blood cancers, because of the predominant presence of NK cells in the peripheral blood and spleen (23). The inventors thus prepared cells which induce an immune response against leukemia lymphocytes.

In their studies, the inventors demonstrated in particular that it was possible to vaccinate mice and protect them against the development of leukemia induced by retroviruses by using tumor cells modified so that they have a negative ERK5 phenotype and a negative MHC-I phenotype. Peptides derived from virus, neutralizing antibodies or immune cells sensitized by an antigen were used in previous studies immediately following infection to block viral replication and prevent the development of a leukemia (32-34). In this study, mice were vaccinated by the inventors with tumor cells modified to present a negative ERK5 phenotype and a negative MHC-I phenotype not loaded with specific antigens, but modified so that they constitute targets for NK cells. Further, the vaccination protocol started two months following infection. To induce a leukemia, the M-MuLV virus must infect mice in the first three days after their birth. It is generally considered that this prevents the generation of an effective immune response against a virus. As a consequence, the protocol employed by the inventors allowed the disease to progress through its first steps and as a consequence inhibition of the immune response against the virus should be effective in these mice. Because EL4 cells are not derived from the M-MuLV virus and are probably not infected by this virus, it is not possible to see that they could induce an immune response against the virus. Further, viral replication is not required at this stage to induce a leukemia. As a consequence, cells expressing shERK5 should induce an immune response against the tumor and play no role in viral infection. The tumor cells modified to present a negative ERK5 phenotype and a negative MHC-I phenotype developed by the inventors are capable of activating an immune response against a variety of tumors. The results presented here show that NK cells play an essential role in the first steps of the vaccination process. Recent results have demonstrated the important role played by NK cells in the adaptive immune response. The role of NK cells could involve an effect linked to dendritic cells (DC) or directly to NK cells.

The present invention relates to a living animal tumor cell presenting a negative MHC-I phenotype for use in therapy, in particular as a vaccine or medicament.

The present invention further relates to an in vitro or ex vivo method for modulating the overall level of expression or the overall quantity of MHC-I proteins present at the surface of a living animal tumor cell, comprising

a) culturing an isolated or cultured animal tumor cell
(i) in a medium which favors the fermentation metabolic pathway at the expense of the respiration metabolic pathway, or
(ii) in a medium which favors the respiration metabolic pathway at the expense of the fermentation metabolic pathway;
for a period of time sufficient to allow the overall level of expression or the overall quantity of MHC-I proteins present at the surface of said tumor cell to be modulated, and
b) recovering said tumor cell.

The period of time for which the cells are cultured is preferably comprised between 2 days and 6 months, for example 3 days to 4 months, 3 days to 1 month or 3 to 12 days.

The favoring of the respiration metabolic pathway at the expense of the fermentation metabolic pathway or the contrary can for example be evidenced using a metabolic substrate in the media, eg glucose, which is marked with radioactive ¹⁴C. The recovery of a high level of ¹⁴C-containing CO₂ signifies the cells mainly use the respiration pathway whereas the recovery of a high level of ¹⁴C-containing lactate signifies the cells mainly use the fermentation pathway. The favoring of the respiration metabolic pathway at the expense of the fermentation metabolic pathway can also be evidenced by analyzing the mitochondrial content by FACS using non acridine orange (NAO) or mitotracker Red (see in particular FIG. 22). The detection of a high level of fluorescence signifies the cells mainly use the respiration pathway.

In a preferred embodiment the medium comprises one or more compounds which favor the fermentation metabolic pathway at the expense of the respiration metabolic pathway, wherein said one or more compounds are present in a quantity sufficient to decrease the overall level of expression or the overall quantity of MHC-I proteins present at the surface of said cell. A medium which favors the fermentation metabolic pathway at the expense of the respiration metabolic pathway is for example a medium containing a fermentative substrate such as glucose, or a compound that blocks respiration such as the mitochondrial inhibitors rotenone, oligomycin, antimycin or carbonylcyanide p-trifluoromethoxyphenylhydrazone (FCCP). In a particularly preferred embodiment, the tumor cells are recovered when they present a reduction of 50% to 100% of the overall level of expression or the overall quantity of MHC-I proteins present at their surface compared with their overall level of expression or their overall quantity in the absence of said one or more compounds. In a particular embodiment, the medium comprises glucose at a concentration ranging from 10 to 100 mM, for example at a concentration of 25 mM.

The method according to this embodiment, can also further comprise the steps of
(i) introducing into said cell an exogenous agent which is capable under appropriate culture conditions of reducing the level of expression or the quantity of the endogenous ERK5 protein in said cell, and optionally which is also capable of reducing the overall level of expression or the overall quantity of MHC-I proteins present at the surface of said cell; and
(ii) culturing said tumor cell under conditions and for a period of time which is sufficient for said cell to present a negative ERK5 phenotype in addition to a negative MHC-I phenotype.

Examples of suitable exogenous agents capable of reducing ERK5 levels include small organic molecules, peptides, intracellular antibodies and nucleic acids as detailed below.

According to another embodiment, the medium comprises one or more compounds which favor the respiration metabolic pathway at the expense of the fermentation metabolic pathway, wherein said one or more compounds are present in a quantity sufficient to increase the overall level of expression or the overall quantity of MHC-I proteins present at the surface of said cell.

A medium which favors the respiration metabolic pathway at the expense of the fermentation metabolic pathway is for example a medium containing glutamine or pyruvate together with galactose, or a pyruvate dehydrogenase kinase (PDK1) inhibitor such as Dichloroacetate (DCA). In a specific embodiment, the tumor cells are recovered when they present an increase of 50% to 100% of the overall level of expression or the overall quantity of MHC-I proteins present at their surface compared with their overall level of expression or their overall quantity in the absence of said one or more compounds. In a particular embodiment, the medium comprises
glutamine at a concentration ranging from to 1 to 10 mM and galactose at a concentration ranging from 1 to 25 mM; or
pyruvate at a concentration ranging from to 1 to 50 mM and malate at a concentration ranging from 1 to 50 mM; or
dichloroacetate at a concentration ranging from 1 to 100 mM.

Tumor cells thus obtained presenting a positive MHC-I phenotype can be used in vitro to activate Cytotoxic T lymphocytes (CTL). These MHC-I positive tumor cells can also be used as a medicament or vaccine to promote a CTL response.

The present invention is also directed to a living tumor cell which can be obtained by the above methods, in particular to MHC negative tumor cells thus obtained.

The present invention further relates to a living animal tumor cell presenting a negative ERK5 phenotype and a negative MHC-I phenotype.

In particular, the present invention concerns a living animal tumor cell comprising one or more agents of exogenous origin which is (are) capable under appropriate culturing conditions of reducing:
the level of expression or the quantity of endogenous ERK5 protein, for example human ERK5 protein, represented by sequence SEQ ID NO: 1 or one of its animal equivalents; and
the overall level of expression or the overall quantity of MHC-I proteins present at the cell surface;

such that the cell has a negative ERK5 phenotype and a negative MHC-I phenotype.

The present invention also concerns a living animal tumor cell presenting a negative ERK5 phenotype and a negative MHC-I phenotype, comprising one or more agents of exogenous origin which is (are) capable under appropriate culturing conditions of reducing:
the level of expression or the quantity of endogenous ERK5 protein (for example human ERK5 protein represented by sequence SEQ ID NO: 1 in the case of a human tumor cell or one of its animal equivalents in the case of an animal tumor cell); and/or
the overall level of expression or the overall quantity of MHC-I proteins present at the cell surface.

Preferably, the cells of the invention are in the isolated form or in culture.

The term “cell presenting a negative ERK5 phenotype” means a cell having a reduction of at least 10%, preferably 25% to 90%, for example 25% to 50% or 50% to 75% in the level of expression or the quantity of ERK5 protein present in the cell, in particular in the mitochondrial fraction, compared with its level of expression or its quantity in the absence of exogenous agent under identical culture conditions. According to a particularly preferred embodiment, the reduction in the level of expression or the quantity of ERK5 protein is not equal to 100%. Preferably again, it is in the range 25% to 50%, in the range 45% to 55% or in the range 50% to 75%. Preferably, for a given type of tumor cell, the percentage reduction in the level of expression or the quantity of ERK5 protein is such that the cells may be maintained in culture for a period of at least one, preferably two, three or six months.

According to the invention, tumour cells are considered as “presenting a negative MHC-I phenotype” when said tumor cells are capabale of being lysed by syngenic NK cells (e.g. cells from the same individual or from a closely related immunologically compatible individual), including by non activated NK cells. The ability to be lysed by syngenic non activated NK cells is thus an indication that a tumor cell has an MHC-I negative phenotype. In the context of the present invention, when at least 40%, preferably at least 50% of tumor cells (T) are lysed in vitro after 4 hours in presence of syngenic NK cells (E) at an E:T ratio of 5:1, such tumor cells are to be considered MHC-I negative.

Further, in cases where the MHC-I negative phenotype is induced by introduction into the cell of an exogenous agent, the cell is considered as “presenting a negative MHC-I phenotype” when the cell has a reduction of 25% to 100%, preferably 50% to 100%, more preferably 75% to 95%, for example 85% to 95% of the overall level of expression or the overall quantity at the cell membrane level of MHC-I proteins present at the cell surface (i.e. at the cell membrane level) compared with their overall level of expression or their overall quantity at the membrane level at the cell surface in the absence of said exogenous agent under identical culture conditions. Preferably, for a given type of tumor cell, the percentage reduction in the overall level of expression or the overall quantity at the cell membrane level of MHC-I proteins is such that the cells may be maintained in culture for a period of at least one, preferably two, three or six months.

The term “agent of exogenous origin” means an agent not present in the cell in the natural state.

The term “reduction in the level of expression or the quantity of endogenous ERK5 protein” means a reduction of at least 10%, preferably 25% to 90%, for example 25% to 50% or 50% to 75% in the level of expression or the quantity of ERK5 protein present in a cell with respect to its level of expression or its quantity in the cell in the absence of exogenous agent under identical culture conditions. According to a particularly preferred embodiment, the reduction in the level of expression or the quantity of ERK5 protein is not equal to 100%. Preferably again, it is in the range 25% to 50%, in the range 45% to 55% or in the range 50% to 75%.

The term “reduction in the overall level of expression or the overall quantity of MHC-I proteins” means a reduction of 25% to 100%, preferably 50% to 100%, more preferably 75% to 95%, for example 85% to 95% in the level of expression or the quantity of all of the proteins expressed at the surface of a cell (membrane proteins) forming part of the proteins of the major histocompatibility complex class I (MHC-I) compared with their level of expression or their quantity at the cell surface in the absence of said exogenous agent under identical culture conditions.

The level of expression or the quantity of ERK5 protein in a cell of the invention may be measured using various techniques which are well known to the skilled person, for example by Western Blot detection of specific ERK5 antibodies.

The overall level of expression or the overall quantity of MHC-I proteins present at the cell surface may be measured using various techniques which are well known to the skilled person, for example by FACS (fluorescence activated cell sorting) type flow cytometry to detect labeled antibodies specific of MHC-I proteins.

The term “animal equivalents” of human ERK5 protein represented by the sequence SEQ ID NO: 1 means proteins of various animal species, for example the mouse, rat, dog, pig, cat or other mammal, having high sequence homology or identity and function with human ERK5 protein as represented in sequence SEQ ID NO: 1 (see FIG. 8), for example a protein having a homology or sequence identity of at least 70%, 75%, 80%, 85%, 90% or 95% with the sequence SEQ ID NO: 1 of human ERK5 protein or a protein coded by a gene having at least 70%, 75%, 80%, 85%, 90% or 95% sequence identity or homology with the sequence of the gene coding for human ERK5 protein (ENTREZ Gene ID: 5598, HGNC: 6880, localization: 17p 11.2), and having the same MAP kinase activity and involved in the same functional cascades thereas. They may in particular be proteins coded by orthologous genes as regards the gene coding for the human ERK5 protein, i.e. genes present in different organisms, having evolved from the same ancestral gene following speciation events.

Appropriate culture conditions which can produce cells presenting a negative ERK5 phenotype and/or a negative MHC-I are determined in particular as a function of the type of tumor cells and the agent or agents used. In particular, in the case of using a single agent consisting of an expression vector of a shRNA type molecule targeting ERK5 in murine T or B lymphocytes, the inventors have established that under normal culture conditions a period of at least two months is necessary for the cells to have a negative ERK5 phenotype and a negative MHC-I phenotype. According to a preferred embodiment, the tumor cells are cultured in a medium which favors the fermentation metabolic pathway at the expense of the respiration metabolic pathway, for example a medium comprising 10 to 100 nM of glucose, for a period of time sufficient to allow the cells to present an MHC-I negative phenotype, for example 3 to 12 days. Preferably, the cell of the invention is a mammalian cell, for example a rat, mouse, dog, pig or cat cell. In a particularly preferred embodiment, it is a human cell or a humanized cell.

The reduction in the level of expression or the quantity of ERK5 protein in the cell and the overall level of expression or the overall quantity of MHC-I proteins present at the cell surface may be obtained using various exogenous agents such as, for example, a small organic molecule having a molecular weight in the range 100 to 2500 Da, a peptide, an intracellular antibody or a molecule of nucleic acid. According to a preferred embodiment of the invention, a single agent, for example a vector directing the synthesis of an interfering RNA molecule targeting the ERK5 protein, is used to obtain a reduction in the level of expression or the quantity of ERK5 protein in the cell and the overall level of expression or the overall quantity of MHC-I proteins present at the cell surface. According to another preferred embodiment of the invention, a combination of two or more agents may be used to obtain this reduction. According to a particular embodiment, a first exogenous agent, for example a vector directing the synthesis of an interfering RNA molecule targeting the ERK5 protein, is used to reduce the level of expression or the quantity of ERK5 protein, and a second, for example a vector directing the synthesis of the adenoviral protein gp19K or a vector directing the synthesis of an interfering RNA molecule targeting β2-microglobulin, is used to reduce the overall level of expression or the overall quantity of MHC-I proteins present at the cell surface. According to another preferred embodiment, an exogenous agent, for example a vector directing the synthesis of an interfering RNA molecule targeting the ERK5 protein, is used to reduce the level of expression or the quantity of ERK5 protein, and the tumor cell is cultured in a fermentative medium for at least 3 to 12 days until it also presents a negative MHC-I phenotype. These embodiments are particularly preferred in the case in which the tumor cells are primary cells obtained from a patient afflicted with a cancer and modified to present a negative ERK5 phenotype and a negative MHC-I phenotype, and intended to be administered to the same patient for therapeutic purposes. As explained above, the time necessary for expression of the negative MHC-I phenotype by cancer cells transformed solely by a vector directing the synthesis of an interfering RNA molecule targeting the ERK5 protein (at least two months for murine T or B lymphocytes) may in some cases prove to have little compatibility with therapeutic use. These embodiments can also be used for certain tumor cells, for example Jukart cells, for which the use of shERK5 alone is not sufficient to obtain cells presenting a negative MHC-I phenotype after five or six months of culture (FIG. 25). According to a preferred embodiment, the reduction in the level of expression or the quantity of ERK5 protein in the cell and in the overall level of expression or the overall quantity of MHC-I proteins present at the cell surface is caused at least in part by a post-transcriptional mechanism. According to this embodiment, the agent of exogenous origin preferably comprises a molecule of nucleic acid of exogenous origin comprising a sequence of 15 to 25, for example 18 to 24, nucleotide residues having a degree of homology of at least 85%, for example at least 90%, at least 95% or 100% with a portion of the nucleotide sequence of the gene or cDNA encoding the human ERK5 protein represented by sequence SEQ ID NO: 2, or one of its animal equivalents.

The term “degree of homology” more particularly means the percentage identity between two DNA or RNA sequences, of complementarity between two DNA or RNA sequences or of equivalence or complementarity between a DNA sequence and a RNA sequence or conversely between a RNA sequence and a DNA sequence.

The molecule of nucleic acid of exogenous origin is preferably a molecule of antisense RNA, sense RNA, miRNA, siRNA or ribozyme type, or a molecule of DNA comprising a sequence the transcription of which generates a RNA molecule of the shRNA, antisense RNA, sense RNA, miRNA, siRNA or ribozyme type, under the control of a promoter which is active in the cell. Preferably, the molecule of nucleic acid of exogenous origin is not a double-stranded RNA with more than 26 base pairs.

The term “shRNA” molecule or “short hairpin RNA” means a RNA strand which comprises a sense sequence (i) preferably comprising 15 to 25 nucleotides, homologous to a sequence of a target DNA or RNA and an antisense sequence (ii) complementary to sequence (i), sequences (i) and (ii) being or not being separated by a sequence (iii) comprising at least 2, for example 2 to 100, 2 to 50, 3 to 40 or 10 to 40 nucleotides. Due to the complementarity of sense (i) and antisense (ii) sequences, these RNA molecules tend to fold upon themselves to take the form of a bicatenary RNA formed as a hairpin comprising a loop constituted by the sequence (iii). Classically, expression of a “shRNA” molecule is under the control of a promoter recognized by a RNA polymerase, for example RNA polymerase II or III, preferably RNA polymerase III, for example the U6 or H1 promoter.

A miRNA (or “micro RNA” or “miR”) is a single-strand RNA with a length which is generally in the range 21 to 24 nucleotides, encoded by a gene (not coding for a protein) which firstly is transcribed into a pri-RNA molecule (primary transcript), which is then transformed by the intervention of Drosha nuclease and the Pasha double-stranded RNA binding protein to generate a short hairpin RNA of about 70 nucleotides (pre-miRNA). The miRNA is then generated by the action of dicer endonucelase on the pre-miRNA in the cytoplasm. The genomes of the majority of pluricellular organisms generally include several hundred micro RNA genes. miRNAs are post-transcriptional repressors: by pairing with messenger RNA, they guide their degradation, or repression of their translation into protein. In particular, it has been shown that “miRNA 143” (GenBank accession number: AJ535834) reduces expression of the ERK5 protein (35).

The term “siRNA” (“short interfering RNA” or “small interfering RNA” or “silencing RNA”) means a short double-stranded RNA with a length of 15 to 25, preferably 18 to 24 base pairs, having at least 85% homology with a sequence of a target DNA or RNA. These molecules are normally capable of provoking a RNA interference mechanism in mammalian cells without triggering the non-specific interferon response.

The term “antisense RNA” more particularly means a RNA molecule comprising a sequence of at least 15, preferably at least 20 or 25, for example 20 to 500, 20 to 200, 25 to 150 or 25 to 100 nucleotides, with a complementarity of at least 85% with the mRNA molecule obtained following transcription of a target gene.

The term “sense RNA” more particularly means a RNA molecule comprising a sequence of at least 15, preferably at least 20 or 25, for example 20 to 500, 20 to 200, 25 to 150 or 25 to 100 nucleotides with at least 85% identity with the mRNA molecule obtained following transcription of a target gene.

The term “ribozyme” more particularly means a ribozyme comprising at least one sequence of at least 15, preferably at least 20 or 25, for example 20 to 500, 20 to 200, 25 to 150 or 25 to 100 nucleotides having at least 85% complementarity with the mRNA molecule obtained following transcription of a target gene and capable of cleaving said mRNA.

According to a particularly preferred embodiment, the reduction in the level of expression or the quantity of ERK5 protein in the cell and the overall level of expression or the overall quantity of MHC-I proteins present at the cell surface is caused at least in part by a RNA interference mechanism. According to a preferred variation of this embodiment, the nucleic acid molecule of exogenous origin is a shRNA molecule. According to a particularly preferred embodiment, the nucleic acid molecule of exogenous origin comprises a molecule of DNA comprising one of sequences SEQ ID NO: 3, 4 or 5 under the control of a promoter which is active in the cell, the transcription of which generates a shRNA molecule.

According to yet another preferred embodiment, the reduction in the overall level of expression or the overall quantity of MHC-I proteins present at the cell, and optionally also that of the level of expression or the quantity of ERK5 protein in the cell is caused at least in part by culturing the cells in a medium which favors the fermentation metabolic pathway at the expense of the respiration metabolic pathway, for example a medium comprising 10 to 100 nM of glucose, for a period of time sufficient to allow the cells to present an MHC-I negative phenotype, for example 3 to 12 days.

The molecule of nucleic acid of exogenous origin is preferably introduced into the cell via a plasmid or viral vector. In particular, it may be an integrative vector, especially a lentiviral or retroviral vector, or a non integrative vector, especially an adenoviral vector. According to a particularly preferred embodiment, the molecule of nucleic acid of exogenous origin is introduced into the cell via a non integrative viral vector such as an adenoviral or aden-associated vector. According to another preferred embodiment, the molecule of nucleic acid of exogenous origin is introduced into the cell via a non viral vector, in particular a vector based on nanoparticles, in particular nanospheres of mesoporous silica to which polyamidoamine (PAMAM) dendrimers are covalently bonded and complexed with a DNA plasmid (36).

According to a preferred embodiment, the tumor cell of the invention is a lymphocyte, a leukocyte, a breast or prostate cancer cell or a metastatic cell.

According to a particularly preferred embodiment, the cell of the invention is a primary tumor cell extracted from a patient afflicted with a tumor, modified by adding said agent of exogenous origin maintained in culture under conditions and for a period which is sufficient to present a negative MHC-I phenotype, and preferably also a negative ERK5 phenotype. According to this embodiment, a second exogenous agent, for example a vector directing the synthesis of the adenoviral protein gp19K or a vector directing the synthesis of an interfering RNA molecule targeting β2-microglobulin, is preferably also used to reduce the overall level of expression or the overall quantity of MHC-I proteins present at the cell surface.

According to another preferred embodiment, the cell of the invention is obtained by culturing a primary tumor cell extracted from a patient afflicted with a tumor in a fermentative culture medium, in particular a medium containing at least 10 mM, preferably at least 25 mM glucose. According to this embodiment, the cell can be further modified by introduction of an exogenous agent, for example a vector directing the synthesis of the adenoviral protein gp19K or a vector directing the synthesis of an interfering RNA molecule targeting ERK5 or 32-microglobulin, to further reduce the overall level of expression or the overall quantity of MHC-I proteins present at the cell surface and/or that of the ERK5 protein.

According to another particularly preferred embodiment, the cell of the invention is a cell from a tumor cell line, modified by adding the agent of exogenous origin and maintained in culture under conditions and for a period sufficient to present a negative ERK5 phenotype and a negative MHC-I phenotype.

The present invention also concerns a tumor cell line obtained from a cell according to the invention.

The present invention also concerns a cell according to the invention, as a vaccine or medicament.

The present invention also concerns a method for treating or preventing a cancer in a patient, comprising administering tumor cells which present a MHC-I negative phenotype and preferably also a negative ERK5 phenotype to a patient in doses, frequencies and for a period determined as a function of the pathology, antecedents and age. Preferably the tumor cells which present a MHC-I negative phenotype and preferably also a negative ERK5 phenotype are prepared according to a method of the present invention.

The cells of the invention may be used in therapy, in particular to prevent or treat a cancer or the development of metastases in a human or animal patient.

In particular, the cells of the invention may be used for the manufacture of a medicament or vaccine intended to prevent or treat a cancer or the development of metastases in a human or animal patient.

Vaccination is a technique consisting in the inoculation of a germ into the organism, for example a bacterium or a weakened or killed virus. As a consequence, this inoculation results in the development of a specific immune response in the organism. Conventionally, vaccines are used in a preventive manner, thus the term “preventive” or “prophylactic” vaccination is used. The specific immune response triggered by such vaccines induces the presence of memory cells (B and T lymphocytes) in the organism, which allow a rapid and effective immune response during a new infection. More recently, new vaccines have been studied to treat for an individual who is already sick. This is then termed “therapeutic vaccination”. Using the same principles of injection of a weakened or killed vaccine, the desired aim here is not to develop a long term memory, but to stimulate the immune system. This type of approach was studied in particular in order to treat diseases in which the immune system is severely compromised, in particular to treat cancers or auto-immune diseases. According to a particularly preferred embodiment, the cells of the invention are used to manufacture a therapeutic vaccine intended to treat a cancer or the development of metastases in a human or animal patient.

The medicament or vaccine preferably contains the cells of the invention in the live form.

Preferably, the patient is a newborn, child or adult human patient.

According to a preferred embodiment, the medicament or vaccine comprises primary tumor cells extracted from the patient to be treated, modified to present an MHC-I negative phenotype, and preferably also an ERK5 negative phenotype. The primary tumor cells can in particular be modified by adding an exogenous agent into the cells and maintaining them in culture under conditions and for a period sufficient for the cells to present a negative MHC-I phenotype and preferably also a negative ERK5 phenotype. Alternatively the primary cell can be cultured in a medium which favors the fermentation metabolic pathway at the expense of the respiration metabolic pathway, for example a medium comprising 10 to 100 nM of glucose, for a period of time sufficient to allow the cells to present an MHC-I negative phenotype, for example 3 to 12 days.

According to another preferred embodiment, the medicament or vaccine comprises allogenic tumor cells deriving from a cell line. According to this embodiment, the tumor cells used to manufacture the medicament or vaccine may be tumor cells of the same type or a different type to those which are responsible for the cancer with which the patient is afflicted.

According to a preferred embodiment, the medicament or vaccine is used to prevent or treat a cancer against which a large number of NK cells can be mobilized, in particular a cancer of the blood or the bone marrow. According to a particularly preferred embodiment, the medicament or vaccine is used to prevent or treat the development of a leukemia, a lymphoma, a myeloma, a breast cancer or a prostate cancer.

Leukemia is a cancer affecting blood cells and is characterized by abnormal and excessive proliferation of precursors of white cells, blocked at a differentiation stage, which finish by completely invading the bone marrow and then the blood. Leukemic cells may also invade other organs such as lymphatic ganglia, the spleen, the liver or the central nervous system. Acute leukemia is characterized by the rapid proliferation of immature, histologically abnormal and ineffective blood cells. They appear in children and young adults; immediate treatment must be carried out to prevent diffusion of these cells through the blood and organs. Chronic leukemia is characterized by more mature cancer cells, albeit still abnormal and passing through the blood, and by a change occurring over months to years. Chronic leukemia appears principally in elderly persons. This type of leukemia can be treated at a later stage.

The medicament or vaccine of the invention may be used to treat the different types of leukemia, in particular acute lymphoid leukemia (ALL), chronic lymphoid leukemia (CLL), acute myeloid leukemia (AML) or chronic myeloid leukemia (CML) at various stages of their evolution.

A lymphoma is a cancer of the lymphatic system. The lymphatic system comprises the bone marrow, the spleen, the thymus, lymphatic ganglia and the blood vessels and provides the organism's defenses. Lymphomas are distinguished from leukemias by the fact that they are tumors which develop in the secondary lymphoid regions.

The medicament or vaccine of the invention may be used to treat the various types of lymphomas, in particular lymphomas of phenotype B, lymphomas of phenotype T and NK and non Hodgkins lymphomas as defined by the WHO (World Heath Organization) classification. Further, the medicament or vaccine of the invention may be used to treat the various types of Hodgkins or non Hodgkins lymphomas at various stages of their evolution, in particular at the four stages distinguished by the Ann Arbor classification (stage I: attack of a single ganglion group or a single organ; stage 11: affliction in more than one ganglion area on the same side of the diaphragm (lower part or upper part of body); stage III: multiple adenopathies on both sides of the diaphragm (lower part and upper part of the body); stage IV: diffuse affliction of one or more viscerae and the bone marrow).

A myeloma is cancer of the bone marrow affecting the plasmocytes (i.e. activated B lymphocytes), and involving the synthesis of an abnormal immunoglobulin. “Multiple myeloma of the bones” or “Kahler's disease” is characterized by the development in the skeleton of multiple osteolytic tumors with plasmocytes (plasmocytomas) which in 80% of cases secrete an immunoglobulin, either of type G (⅔ of cases) or of type A (⅓ of cases) which gradually destroy the adjacent bone.

The medicament or vaccine of the invention may be used to treat the various types of myelomas, in particular multiple myelomas of the bones. Further, the medicament or vaccine of the invention may be used to treat the various types of myelomas at various stages in their evolution, in particular in the three stages distinguished by the Durie-Salmon classification (stage I: small tumor mass, less than 600 billion malignant cells/m²; stage II: intermediate tumoral mass, between 600 and 1200 billion malignant cells/m²; stage III: large tumoral mass, more than 1200 billion malignant cells/m²), as well as at the various sub-classification stages which are a function of the renal function (stage A: correct renal function, creatininemia less than 20 mg/l or 180 μmol/l; stage B: deteriorated renal function, creatininemia more than 20 mg/l or 180 μmol/l) and of the type of bone lesions (0=no bone lesions; 1=osteoporosis; 2=bone lysis lesions; 3=major bone lesions and fractures).

According to a preferred embodiment, the medicament or vaccine is used to prevent or treat the development of metastases.

According to another preferred embodiment, the tumor cells used for the manufacture of the medicament or vaccine of the invention are lymphocytes, in particular B or T lymphocytes.

The vaccine or medicament of the invention may be administered in doses, frequencies and for periods determined as a function of the pathology of the patient, their antecedents and age. The vaccine or medicament of the invention may be administered to the patient from diagnosis of the cancer and until cure. In particular, it may be administered at various stages in the development of cancer. Preferably, the vaccine or medicament of the invention is administered to a human patient in doses comprising at least 1 000 000, preferably between 1 000 000 and 50 000 000, for example between 5 000 000 and 20 000 000 cells of the invention and at daily, weekly, bimonthly or monthly intervals.

The vaccine or medicament of the invention may, for example, be administered to the patient intravenously, intradermally, intramuscularly, intraperitoneally or subcutaneously. Preferably, the vaccine or medicament of the invention is administered locally in the near vicinity of “endogenous” tumor cells of the patient or in the near vicinity of or into the organ of the patient afflicted with the cancer or into the blood system.

According to a preferred embodiment, the vaccine or medicament of the invention is intended to be administered to the patient in combination with an allogenic transplantation of bone marrow or hematopoietic stem cells.

The HLA (human leukocyte antigen) system which corresponds to the human major histocompatiblity complex, allows the body and its immune system to recognize self (all tissues, etc), from non-self (virus, bacteria, grafts). Each individual possesses a unique HLA type which is found on the cell surface. Thus, any cell which does not have self HLA markers on its surface is attacked by the immune system. HLA typing of an individual is determined by the various alleles which represent the 6 genes A, B, C, DR, DQ and DP governing histocompatiblity. These genes are all present on chromosome No 6 and include many alleles. Indeed, 268 alleles have been identified in gene A, 517 for gene B, 129 for gene C, 333 for gene DR 53 for gene DQ and 109 for gene DP. Because of the large number of alleles for each of these genes, the number of possible combinations is extremely high.

Transplanting allogenic hematopoietic stem cells deriving from a sibling with an identical HLA typing may cure the leukemia, however 75% of patients do not have a brother or sister having an identical HLA type. In this case, a volunteer donor may be used who has a HLA type which is as close as possible to that of the receiver in order to prevent the donor cells from triggering a rejection reaction. In the context of a bone marrow transplant, the immune system of the receiver is greatly weakened or non-existent and thus will not normally be at the origin of a rejection reaction, Cells grafted from the donor, however, which have to produce the new immune system of the patient, may attack the tissues of the receiver, perceived as “non self”. This is termed graft versus host disease, “GvH disease”.

An alternative choice is to use stem cells deriving from members of the family having one haplotype (half of the genotype of an individual deriving either from the father or from the mother) which is identical and one haplotype which is different from that of the receiver. This is termed haplo-identical transplantation. Haplo-identical transplantations must as a consequence be extensively depleted in T lymphocytes to prevent fatal GvH rejection reactions. The anti-leukemic effect of this type of graft cannot, however, rest on the T lymphocytes of the donor, but only on triggering an alloreactive reaction dependent on NK cells of the donor and based on non-self recognition. The alloreactive NK cells will be localized at lympho-hematopoietic sites and attack the lympho-hematopoietic cells of the receiver, including leukemic cells, while sparing other healthy organs. In addition to eliminating leukemic cells, the fact that NK cells kill T lymphocytes of the receiver means that a host versus graft type (HvG) rejection reaction can be avoided and also the fact that NK cells kill dendritic cells of the receiver means that activation of T cells of the donor can be prevented and as a consequence a GvH type rejection reaction can be avoided. This approach, however, can not be used in all cases since about 30% of the population is resistant to the effect of alloreactive NK cells.

The use of the tumor cells of the invention and/or NK cells activated in vitro or ex vivo by the tumor cells of the invention in combination with a bone marrow graft or stem cells, more particularly in the absence of a donor having an identical HLA type, may render the action of NK cells in vivo more effective.

According to another preferred embodiment, the vaccine or medicament of the invention is intended for administration in combination with another anti-cancer treatment, for example a treatment by chemotherapy or radiotherapy.

In particular, the vaccine or medicament of the invention may be intended for administration in combination with a treatment aimed at reinforcing the acquired immune response, for example in combination with transplantation of dendritic cells loaded with tumor antigens.

According to another preferred embodiment, the vaccine or medicament of the invention is intended for administration in combination with the administration of activated NK cells, i.e. NK cells which have already been brought into contact with tumor cells and which have been shown to be capable of causing their lysis or apoptosis, in particular NK cells which have been brought into contact with tumor cells having a negative MHC-I phenotype and optionally also a negative ERK5 phenotype.

The term “administration in combination” means administration of the vaccine or medicament of the invention being carried out simultaneously or separately in time with another anti-cancer treatment.

The present invention also concerns a therapeutic or vaccine composition comprising a cell of the invention as well as a pharmaceutically acceptable vehicle.

According to a particular embodiment, the therapeutic or vaccine composition of the invention also comprises a therapeutic anti-tumoral molecule.

According to a particular embodiment, the therapeutic or vaccine composition of the invention also comprises activated NK cells.

The present invention also pertains to the use of an agent which is capable of endowing a human or animal tumor cell with a negative ERK5 phenotype and a negative MHC-I phenotype, for the manufacture of a living cellular vaccine or medicament intended to prevent or treat a cancer in a human or animal patient.

According to a preferred embodiment, the agent used to manufacture the cellular vaccine or medicament of the invention is capable of:
reducing by at least 10%, preferably 25% to 90%, preferably 25% to 90%, for example 25% to 50% or 50% to 75%, the level of expression or the quantity of ERK5 protein in the tumor cell compared with its level of expression or its quantity in the absence of said agent under identical culture conditions; and
reducing by 50% to 100%, preferably 75% to 95%, for example 85% to 95%, the overall level of expression or the overall quantity of MHC-I proteins present at the surface of the tumor cell compared with their overall level of expression or their overall quantity at the cell surface in the absence of said agent under identical culture conditions.

According to a preferred embodiment, the agent of the invention comprises a molecule of nucleic acid of exogenous origin comprising a sequence of 15 to 25, for example 18 to 24, nucleotide residues having a degree of homology of at least 85%, for example at least 90%, at least 95% or 100% with a portion of the nucleotide sequence of the gene or the cDNA encoding the human ERK5 protein represented by the sequence SEQ ID NO: 2, or one of its animal equivalents, or one of its animal equivalents. The molecule of nucleic acid of exogenous origin is preferably a molecule of the RNA antisense, RNA sense, miRNA, siRNA or ribozyme type, or a DNA molecule comprising a sequence the transcription of which generates a RNA molecule of the shRNA, antisense RNA, sense RNA, miRNA, siRNA or ribozyme type, under the control of a promoter which is active in the cell. According to a particularly preferred embodiment, the molecule of nucleic acid of exogenous origin comprises a molecule of DNA comprising one of sequences SEQ ID NO: No 3, 4 or 5 under the control of a promoter which is active in the cell, the transcription of which generates a shRNA molecule.

According to a preferred embodiment, the agent comprising the nucleic acid molecule of exogenous origin is a plasmid or viral vector, more preferably an integrative viral vector, for example a lentiviral or retroviral vector, and particularly preferably a non integrative viral vector, for example an adenoviral vector.

According to a preferred embodiment, the agent comprising the molecule of nucleic acid of exogenous origin is a vector based on nanoparticles, in particular an agent in which the nucleic acid molecule is included in a DNA plasmid complexed with nanospheres of mesoporous silica to which dendrimers of polyamidoamine (or “PAMAM”) are covalently bonded.

The present invention also pertains to a living animal tumor cell having a negative MHC-I phenotype as a medicament or vaccine. According to a preferred embodiment, the tumor cell also has a negative ERK5 phenotype. The present invention also pertains to an in vitro or ex vivo method for obtaining an animal tumor cell presenting a negative ERK5 phenotype and a negative MHC-I phenotype, comprising:
a) introducing into an isolated or cultured animal cell one or more exogenous agent(s) which are capable under appropriate culture conditions of reducing the level of expression or the quantity of the endogenous ERK5 protein and the overall level of expression or the overall quantity of MHC-I proteins present at the cell surface;
b) culturing the tumor cell under conditions and for a period which are sufficient for it to present a negative ERK5 phenotype and a negative MHC-I phenotype; and
c) recovering said cell.

According to a particularly preferred embodiment of the method of the invention, step a) comprises administering a single exogenous agent, for example a vector directing the synthesis of an interfering RNA molecule targeting the ERK5 protein, capable under appropriate culture conditions of reducing the level of expression or the quantity of ERK5 protein and of reducing the overall level of expression or the overall quantity of MHC-I proteins present at the cell surface. According to another particularly preferred embodiment of the method of the invention, step a) comprises administering a first exogenous agent, for example a vector directing the synthesis of an interfering RNA molecule targeting the ERK5 protein, capable under appropriate culture conditions of reducing the level of expression or the quantity of the ERK5 protein, and a second exogenous agent, for example a vector directing synthesis of the adenoviral protein gp19K or a vector directing the synthesis of an interfering RNA molecule targeting β2-microglobulin, capable under appropriate culture conditions of reducing the overall level of expression or the overall quantity of MHC-I proteins present at the cell surface.

The appropriate culture conditions which can produce cells having a negative ERK5 phenotype and a negative MHC-I phenotype are determined in particular as a function of the type of tumor cells and the agent or agents used. in particular, in the case of using a single agent consisting of an expression vector of a shRNA type molecule targeting ERK5 in murine T or B lymphocytes, the inventors have established that a duration of at least two months is necessary for the cells to present a negative ERK5 phenotype and a negative MHC-I phenotype. This period of time can be shortened by culturing the tumor cells in a medium which favors the fermentation metabolic pathway at the expense of the respiration metabolic pathway. In a specific embodiment, step b) comprises culturing the tumor cells in a medium which favors the fermentation metabolic pathway at the expense of the respiration metabolic pathway, for example a medium comprising 10 to 100 nM of glucose, for a period of time sufficient to allow the cells to present an MHC-I negative phenotype, for example 3 to 12 days. According to a preferred<d embodiment, the tumor cells are recovered when:
a reduction of 10%, preferably 25% to 90%, for example 25% to 50% or 50% to 75%, in the level of expression or the quantity of ERK5 protein present in said cell compared with its level of expression or its quantity in the absence of said exogenous agent under identical culture conditions is obtained; and
a reduction of 50% to 100%, preferably 75% to 95%, for example 85% to 95%, in the overall level of expression or the overall quantity of MHC-I proteins present at the cell surface compared with their overall level of expression or their overall quantity at the cell surface in the absence of said exogenous agent under identical culture conditions is obtained.

Preferably also, the tumor cells are recovered when their MHC-I phenotype is such that at least 40%, preferably at least 50% of tumor cells (T) are lysed in vitro after 4 hours in presence of syngenic NK cells (E) at an E:T ratio of 5:1.

According to a particularly preferred embodiment, the culture conditions and durations are selected so that the reduction in the expression or quantity of the ERK5 protein in the tumor cell obtained by carrying out the method of the invention is not equal to 100%.

According to a preferred embodiment of the method of the invention, the tumor cell mentioned in step a) is a primary cell obtained from a patient afflicted with a cancer.

According to an alternative preferred embodiment of the method of the invention, the tumor cell mentioned in step a) is a cell obtained from a tumor cell line.

The present invention is also directed to activated NK cells, their preparation and their use in therapy. Indeed, the inventors have shown in particular, that 48 h after the injection of L1210-shERK5 cells (which are ERK5 and MHC-I negative) in the peritoneum of mice, the percentage of cells expressing granzymes A and/or B (i.e. activated NK cells) among peritoneal cells was much higher than in the case of the injection of non modified L1210 cells (FIG. 15). MHC-I negative cells can thus also be used in vitro to activate naïve NK cells. The activated NK cells thus obtained can then be administered to a patient.

The present invention thus pertains to an in vitro or ex vivo method for obtaining activated NK cells, comprising:
a) in vitro or ex vivo contacting living NK cells with living tumor cells presenting a negative MHC-I phenotype under conditions and for a period sufficient to induce activation of the NK cells;
b) recovering the activated NK cells.

Preferably the living tumor cells of step a) also present a negative ERK5 phenotype.

More particularly, the term “activated NK cells” means NK cells expressing granzymes A and/or B, FasL and/or perforin, for example as evidenced using FACS. The levels of NK cell activation obtained using the methods of the present invention are significantly higher than levels which might be obtained when the activation is effected by contact of the NK cells with cytokines (for example IL2, IL-12 or IL-15) or with alpha- or beta-interferons.

The culture conditions and period mentioned in step a) of the method of the invention are determined in particular as a function of the types of tumor cells employed. Preferably, NK(E) cells and the tumor cells of the invention (T) are brought into presence of each other at an E:T ratio of at least 1:1, preferably at least 5:1, for example a ratio in the range 5:1 to 35:1, or in the range 5:1 to 15:1, for a period of several hours, preferably at least 5, 7 or 10 hours, for example for a period in the range 5 to 15 hours or between 7 and 10 hours.

The activated NK cells obtained by carrying out the method of the invention may in particular be purified by FACS and preserved under suitable conditions, for example by freezing or maintained in culture until use.

According to a preferred embodiment of the method of the invention, the living tumor cells presenting a negative MHC-I phenotype and optionally a negative ERK5 phenotype are cells of the invention or cells obtained by carrying out the method to obtain the negative ERK5 and MHC-I cells mentioned above.

According to a particular embodiment of the method of the invention, the NK cells mentioned in step a) are primary NK cells obtained from a patient afflicted with a cancer. According to another particular embodiment, the NK cells mentioned in step a) are obtained from a healthy donor. According to another embodiment, the NK cells mentioned in step a) are derived from an NK cell line or from a biological material bank, such as in particular an umbilical cord blood transplant bank, a tissue bank or a blood bank. Preferably the NK cells are mammalian cells, in particular rat, mouse, dog, pig or cat cells. Most preferably the NK cells are human or humanized cells. Preferably still, the living tumor cells presenting a negative MHC-I phenotype are cells obtained according to one of the methods described in the context of the present invention for obtaining such. The present invention is also directed to an activated NK cell which can be obtained by the above method according to the invention.

The present invention is further directed to an activated NK cell which has an activated phenotype. In particular the activated NK cells according to the invention are more efficient in targeting and lysing tumor cells, whether in vitro or in vivo, than NK cells activated in vitro by cytokines (for example by IL2, IL12 or IL15) or by alpha or beta Interferon.

The activated NK according to the invention may be used to manufacture a medicament or vaccine to prevent or treat a cancer. According to a particular embodiment, the medicament or vaccine of the invention also comprises tumor cells of the invention. According to another preferred embodiment, the vaccine or medicament of the invention also comprises dendritic cells loaded with tumor antigens. In fact, a combined graft of NK cells activated by the tumor cells of the invention and dendritic cells loaded with tumor antigens reinforces the anti-tumoral effect.

### FIGURES

FIG. 1. Peritoneal clearance of EL4-shERK5 cells three days following injection. One million cells respectively of EL4-wt, EL4-shERK5-A, EL4-shERK5-B or EL4-shLuc cells, labelled with CFSE (5,6-carboxyfluorescein diacetate succinimidyl ester) were injected into the peritoneum of syngenic C57/B6 mice. Three days later, the peritoneal cells were recovered and survival of the injected cells was evaluated by FACS (fluorescence-activated cell sorting) type flow cytometry. The results show that three days following injection, EL4-wt and EL4-shLuc cells subsisted in the peritoneum, while the EL4-shERK5 cells had been eliminated.

FIG. 2. Surface expression of MHC-I for EL4-shERK5 cells. A) EL4-shERK5-A, EL4-shERK5-B and EL4-shLuc cells were cultivated in the presence or in the absence of a selection antibiotic (puromycin). The cells were labeled with antiHdkb antibodies and the level of expression of MHC-I was analyzed by FACS type flow cytometry. EL4-wt and RMAS cells were used as the positive and negative controls respectively for MHC-I surface expression. The results show that the EL4-shERK5-A and EL4-shERK5-B cells show a loss of surface expression of MHC-I in the presence of the antibiotic. B) Expression of ERK5 by the various cell lines described in A) was analyzed by immunoblotting. Antibodies for Vav and ERK2 were used as controls. The results show that the EL4-shERK5-A and EL4-shERK5-B cells showed a loss of ERK5 expression in the presence of the antibiotic.

FIG. 3. Involvement of NK cells in the elimination of EL4-shERK5 cells. A) Total splenocytes, purified NK cells or splenocytes lacking NK cells were incubated with EL4-shERK5 target cells loaded with ⁵¹Cr in different ratios. After 4 hours, the supernatants were collected and the cytolytic activity was measured as a function of the liberation of ⁵¹Cr. 100% of the EL4-shERK5 cells were lysed in the presence of purified NK cells at NK cell:target cell (E:T) ratios of 35:1 to 75:1. B) Purified NK cells were incubated with EL4-shERK5 or Yac-1 cells labeled with ⁵¹Cr for four hours at different ratios. The cytolytic activity was measured. A much larger number of EL4-shERK5 target cells than Yac-1 target cells were lysed by the NK cells. The results show that NK cells are responsible for the elimination of EL4-shERK5 cells, and that the negative ERK5 phenotype of the target cells leads to an additional effect compared with the negative MHC-I phenotype alone.

FIG. 4. Constitutive activation of ERK5 induces a reporter gene under the control of a MHC-I sensitive promoter. A) Ten million Jurkat cells (human tumor cells) were transferred with 5 μg of the ERK5, MEK5D or ERK5 and MEK5D expression products, 2.5 μg of the reporter gene under the control of a MHC-I gene promoter: Pd-1 and 1 μg of β-galactosdiase expression plasmid (control). 48 hours later, luciferase and galactosidase activities were measured and the results were expressed as the luciferase/galactosidase ratio (relative luciferase activity). The results show that the over-expression of ERK5 coupled with the constitutively active gene MEK5 activates the reporter gene under the control of the MHC-I promoter. B) The experiment was repeated with the respective expression plasmids ERK5 and MEK5D, ERK5KM (an inactive ERK5 mutant), ERK5 AEF (a non activatable ERK5 mutant) or shERK5. The results show that ERK5 induces activation of a MHC-I promoter. Further, blocking the activity of ERK5 by using an inactive mutant (ERK5KM) or reducing its expression with shERK5 reduces the basal level of expression of this promoter. ERK5 thus controls the transcription of MHC-I genes.

FIG. 5. Recruitment of NK cells in the peritoneum after EL4-shERK5 injection in mice. 5×10⁵ EL4-shERK5 or EL4-shLuc or PBS cells were injected into the peritoneum of syngenic mice. Three days later, cell populations from the peritoneum were analyzed by flow cytometry (FACSCalibur). A) Percentage of lymphocytes in the peritoneum. B) Percentage of NK cells recovered in the peritoneum. C) Percentage of NK cells recovered in the lymphocyte population of the peritoneum. The results demonstrate that after injection of EL4-shERK5 cells, NK cells are recruited in the peritoneum.

FIG. 6. Survival of wild type (wt) EL4 cells after co-injection with EL4-shERK5 cells. 2.5×10⁵ EL4-shLuc cells, EL4-shERK5 cells or a mixture of 2.5×10⁵ EL4-shLuc cells and 2.5×10⁵ EL4-shERK5 cells loaded with CFSE were injected into the peritoneum of syngenic mice. The percentage of CFSE+ cells after peritoneum washing three days later is indicated. The EL4-shERK5 cells were completely eliminated while survival of the EL4-shLuc cells was substantially reduced after co-injection with EL4-shERK5 cells.

FIG. 7. Immunization with EL4-shERK5 cells. Mice were immunized over 2 or 6 weeks with 2.5×10⁵ EL4-shERK5 cells and then injected subcutaneously with 2.5×10⁵ EL4-wt cells. Tumor size was measured after 12, 14 and 16 days. The results show that the development of subcutaneous tumors from EL4-wt cells started later and that the tumor volume was greatly diminished in mice immunized over two or six weeks with EL4-shERK5 cells. Immunization with EL4-shERK5 cells thus protects against subcutaneous tumors induced by wild type (wt) EL4 cells.

FIG. 8. Human ERK5 protein. Amino acid sequence of human ERK5 protein (accession no: AAA81381, Zhou et al, 1995) (SEQ ID NO: 1) and nucleotide sequence of cDNA coding for the human ERK5 protein (accession no: U25278, Zhou et al, 1995, coding sequence: nucleotides 84 to 2531) (SEQ ID NO: 2).

FIG. 9. Nucleotide sequences (1), (2) and (3) used for the preparation of expression vectors of “shERK5” molecules (SEQ ID NO: 3, 4 and 5).

FIG. 10. L1210-shERK5 and shLuc cells were loaded with various concentrations of CFSE then injected into the peritoneum of syngenic Balb/c mice which had been treated with rabbit serum (control) or with anti-asialo GM1 antiserum. 48 hours later, the peritoneal cells were analyzed by FACS. The shLuc cells were not eliminated, while the shERK5 cells were eliminated from the peritoneum of the control mice but not from the peritoneum of mice which had received a treatment with the anti-asialo GM1 antiserum which was intended to eliminate NK cells.

FIG. 11. L1210-shERK5 cells were loaded with ³H-thymidine and incubated in the presence or absence of NK cells; (A) syngenic Balb/c or (B) allogenic C57/B6, and L1210-shLuc cells were loaded with ³H-thymidine and incubated in the presence or absence of syngenic Balb/c NK cells (C). The results show that the two types of NK cells (syngenic Balb/c and allogenic C57/B6) recognize L1210-shERK5 cells as foreign and eliminate them with similar efficiency. The L1210-shLuc cells, in contrast, are not recognized as foreign; these cells express MHC-I and are not eliminated. E:T=ratio of effector cells (NK)/target cells (L1210-shERK5).

FIG. 12. L1210-shERK5 cells were loaded with ³H-thymidine and incubated with syngenic Balb/c NK cells in the presence or absence (A) of EGTA (which blocks cell death induced by granzymes) or (B) of an anti-FasL antibody (which blocks cell death induced by Fas). The results show that the quantity of L1210-shERK5 cells eliminated by the NK cells is reduced in the presence of EGTA or anti-FasL antibody, and thus demonstrates the importance of mechanisms employing granzymes and Fas when eliminating shERK5 cells by NK cells.

FIG. 13. The level of expression of MHC-I was analyzed by FACS with an anti-H2 Kd antibody in L1210-shLuc or shERK5 cells. The dotted lines correspond to cells labeled with a control IgG. The results show that the shERK5 cells lose MHC-I expression at the plasmid membrane level.

FIG. 14. Syngenic Balb/c mice received injections of PBS, L1210-shLuc cells or L1210-shERK5 cells into the peritoneum. 48 hours later, the peritoneal cells were recovered and the NK cells were quantified with anti-CD49b antibody. The results show that (B) the number as well as (A) the proportion of NK cells in the peritoneum is higher in mice which had received the shERK5 cells, thus demonstrating that NK cells are recruited in the peritoneum by shERK5 cells.

FIG. 15. Syngenic Balb/c mice received injections of PBS, L1210-shLuc cells or L1210-shERK5 cells in the peritoneum. 48 hours later, the peritoneal cells were recovered and the number of NK cells (CD49b+) expressing A and B granzymes was analyzed by FACS. The results demonstrate that NK cells recruited by L1210-shERK5 cells are positive for granzymes and as a consequence have an activated phenotype.

FIG. 16. L1210-shLuc or L1210-shERK5 cells were incubated for one hour with cytotoxic BM3.3 lymphocytes activated or not activated by a PMA/ionomycin treatment, labeled with an anti-Fas antibody and analyzed by FACS. The results show that the activated T lymphocytes induce a large increase in the level of expression of the Fas death receptor in L1210-shERK5 cells. This phenomenon contributes to explaining why these cells are highly sensitive to attacks by cytotoxic lymphocytes in general.

FIG. 17. The expression of two isoforms L and S of the anti-apoptotic protein c-FLIP was analyzed by immunoblot in L1210-shLuc and L1210-shERK5 cells. The results show that shERK5 cells have reduced levels of the two isoforms of the c-FLIP protein (β-actin: control).

FIG. 18. RNA from L1210-shLuc (control) and L1210-shERK5 cells was isolated and underwent qPCR with specific primers for beta-2-microglobulin. The results indicate that the L1210-shERK5 cells show a great reduction in beta-2-microglobulin, a protein which is essential for stabilization of MHC-I molecules at the plasmid membrane level, which contributes to explaining why L1210-shERK5 cells express very little MHC-I protein at the plasmid membrane level.

FIG. 19. Plasma membrane expression of MHC-I depends on the metabolic status of the cell. A) 30×10³ (25 mM glucose) or 120×10³ (4 mM Gln plus 10 mM galactose) L1210 cells were incubated in the corresponding media for different periods of time. MHC-I expression was analyzed by FACs using an anti-H-2K^{d} antibody. B) L1210 cells were incubated for 3 days in glucose, Gln plus galactose (10 mM) or pyuvate plus malate (12.5 mM each) and MHC-I or CD19 expression analyzed by FACs. C) 30×10³ (25 mM glucose) or 120×10³ (10 mM galactose or 12.5 mM each pyruvate and malate) Jurkat cells were incubated in the corresponding media for different periods of time. MHC-I expression was analyzed 5 days later by FACs using an anti-HLA-ABC antibody. D) 30×10³ L1210 cells were incubated in 25 mM glucose with or without 25 mM DCA. E) L1210 cells were incubated for 3 days in Gln media before being placed in either glucose or Gln media for 3 days and MHC-I expression analyzed by FACs. Staining of cells continuously growing in glucose was identical to that of cells growing in Gln and then in glucose for three days and is not depicted in the figure.

FIG. 20. A) Jurkat cells were incubated for 5 days in glucose or Glutamine media and HLA or MICA expression analyzed by FACs. B) L1210 cells were incubated for 3 days in glucose with or without DCA, and CD19 expression analyzed by FACs. C) Jurkat cells were incubated for 5 days in Gln media. Then, cells were incubated in either glucose or Gln media for 5 days and MHC-I expression analyzed by FACs. Staining of cells continuously growing in glucose was identical to that of cells growing in Gln and then in glucose for three days and is not depicted in the figure.

FIG. 21. Respiration induces expression of class I molecules. A) One (25 mM glucose) or ten (10 mM galactose or 12.5 mM each pyruvate and malate) million L1210 cells were grown in the different media for 3 days. RNA was extracted and analyzed by qPCR with specific primers for β₂m (left) or H-2 Kd (right). B) Ten million Jurkat cells were transfected with 2.5 μg of the reporter gene PD-1-Luc and 1 μg of the β-galactosidase expression vector. One day later cells were placed in media containing glucose, Gln or pyruvate plus malate with or without DCA and 2-deoxyglucose (5 mM). Forty-eight hours later, lysates were prepared and analyzed for luciferase and galactosidase activity. The relative luciferase units represent the ratio of luciferase/galactosidase. The data are presented as the mean±SD of at least 3 independent experiments, and were evaluated using Student's t test: * p<0.05; **p<0.005 compared to control cells. C) L1210 cells were incubated for 3 days in glucose, Gln and pyuvate plus malate media and MHC-I expression analyzed by FACs in intact (left) or permeabilized (right) panel.

FIG. 22. A) Ten million Jurkat cells were transfected with 2.5 μg of the reporter gene HLA-A-Luc and 1 μg of the b-galactosidase expression vector. One day later cells were placed in media containing glucose (25 mM) or Gln (4 mM) plus galactose (10 mM). Forty-eight hours later, lysates were prepared and analyzed for luciferase and galactosidase activity. The relative luciferase units represent the ratio of luciferase/galactosidase. The data are presented as the mean±SD of 3 independent experiments, and were evaluated using Student's t test: ** p<0.001 compared to control cells. B) One million Jurkat cells were placed in media containing glucose (25 mM) or Gln (4 mM) plus galactose (10 mM). Five days later, mitochondrial content was analyzed by FACs using nonyl acridine orange (NAO) or mitotracker Red. C) L1210 cells were incubated for 3 days in Gln media. Then, cells were incubated in either glucose or Gln media for 3 days and protein expression wascanalyzed by western blot. D) Downregulated ERK5 expression in shERK5-expressing cells. Protein expression in whole cell extracts was analyzed by western blotting with the corresponding antibodies.

FIG. 23. Respiration induces ERK5 expression. A) One (glucose) or two (Gln and pyruvate plus malate) million cells were incubated in the different culture media for three days and expression of different proteins reveal by immunoblotting. B) Ten million Jurkat cells were transfected with the following: 5 μg of expression vectors for wt ERK5, constituively active MEK5 (MEK5D), 2.5 μg of the reporter gene PD-1-Luc, and 1 μg of the β-galactosidase expression vector. One day later cells were placed in media containing glucose (25 mM), 2-deoxyglucose (2 mM), DCA (15 mM), Gln (4 mM) and/or pyruvate plus malate (12.5 mM each). Forty-eight hours later, lysates were prepared and analyzed for luciferase and galactosidase activity. The relative luciferase units represent the ratio of luciferase/galactosidase. The data are presented as the mean±SD of at least 3 independent experiments, and were evaluated using Student's t test: * p<0.05; ** p<0.005 compared to control cells

FIG. 24. ERK5 localizes in mitochondria in leukemic cells. A) Left panel: one million jurkat cells were fixed with paraformaldehyde and stained with mitotracker red. ERK5 and Hoescht. Cells were analyzed by confocal microscopy. Right panel: Jurkat cell extracts were fractionated in mitochondria (m) and cytosol (c) fractions to investigate the relative localization of different proteins. Fifty μg of these fractions or whole cell extract were analyzed for ERK5 localization. The purity of the fractions was analyzed using Lat, Topoisomerase and Cox IV as markers of cytosol, nuclei and mitochondria respectively. B) Respiration induces ERK5 translocation to mitochondria. L1210 cells were incubated for three days in glucose or Gln media and stained as in A) but Cox IV (in red) was used to label mitochondria (lower panel) or subjected to subcellular fractionation as in A). C) Downregulation of ERK5 expression impairs cell survival in OXPHOS media. Fifty (glucose) or 150×10³ (OXPHOS) L1210 or Jurkat cells were incubated for 72 hours in the different media. Survival was assayed as described in Materials and Methods. The data are presented as the mean±SD of at least 3 independent experiments, and were evaluated using Student's t test: * p<0.005; ** p<0.0005 compared to control cells.

FIG. 25. Jurkat wt cells or expressing small hairpin RNAs against ERK5 (shERK5, cell lines 3 and 4) or a scramble sequence (shscr) were analyzed for HLA expression. The correct downregulation of ERK5 protein was analyzed by western bloting using actin as loading control.

### EXPERIMENTAL SECTION

### Example 1

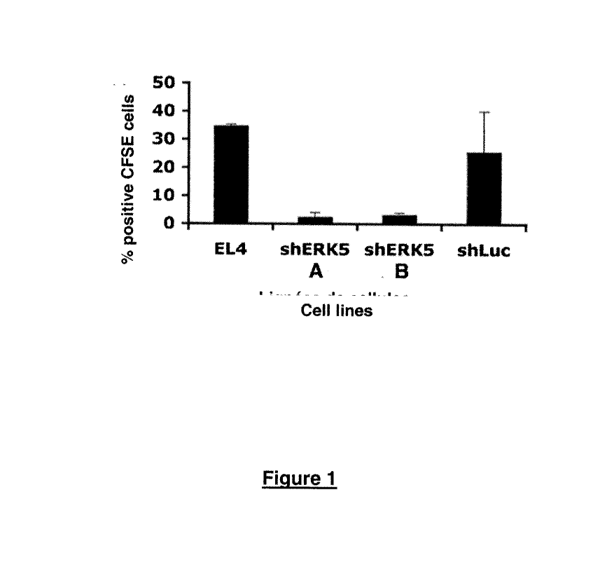

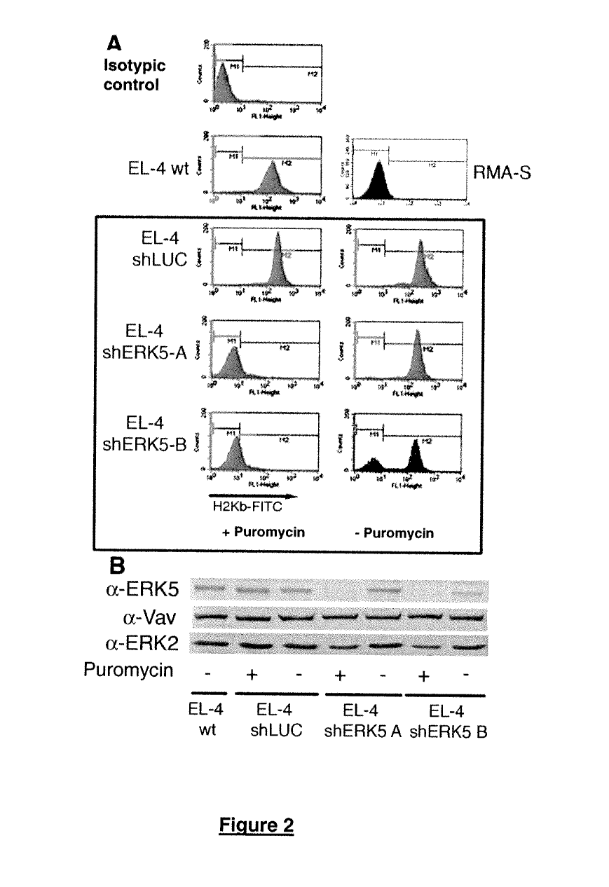

The inventors have recently shown that EL4 cells expressing a shERK5 (EL4-shERK5) could not be caused to induce subcutaneous tumors in syngenic mice (21). These cells were also not caused to induce tumors when injected into the peritoneum, where they were eliminated in less than 72 h following injection (FIG. 1). EL4 cells expressing a luciferase short hairpin RNA (shRNA) (EL4-shLuc) were recovered in amounts similar to those of the wild type (wt) cells, in agreement with the results obtained previously by the inventors with subcutaneous tumors (21). This short period for clearance of cancer cells suggests that the innate immune response, i.e. NK cells, is at the origin of the immune response against these cells. In the same manner, EL4-shERK5 cells but not EL4-shLuc cells reduced the surface expression of MHC-I (FIG. 2A). The EL4-shERK5 cells expressed amounts of MHC-I similar to those of RMA-S cells considered to be negative MHC-I (29). This explains why NK cells recognized EL4-shERK5 cells as “foreign”. The reduction in the level of expression of MHC-I was observed after several weeks in antibiotic selections. In order to determine whether this effect was reversible, the inventors incubated EL4-shERK5 cells in the absence of the antibiotic used for selection. After several weeks, these cells started to recover normal levels of the protein ERK5 (FIG. 2B) and in contrast they recovered expression of MHC-I (FIG. 2A). When the inventors used the same protocol with cells expressing shLuc, they did not observe a change in the level of expression of MHC-I or ERK5.

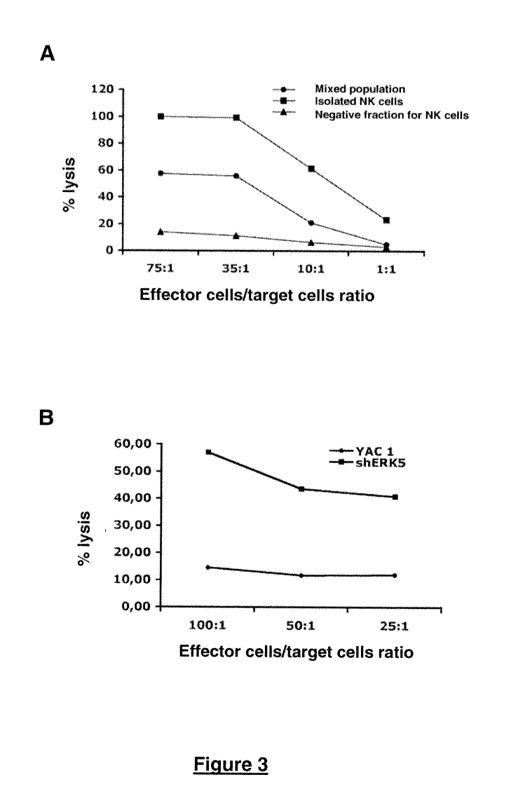

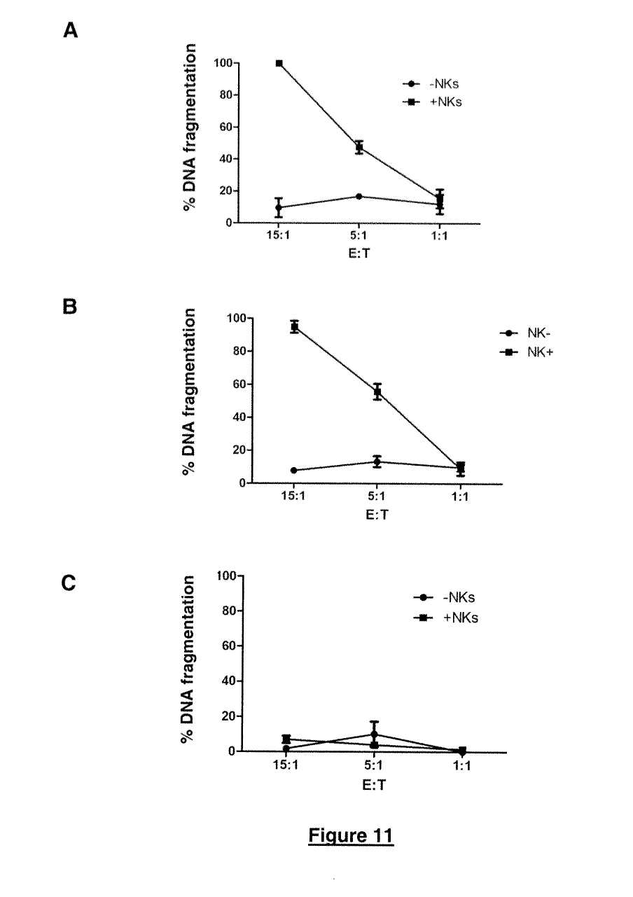

The inventors then used EL4-shERK5 cells as targets for three different populations of effectors: total splenocytes, purified NK cells and splenocytes from which NK cells had been removed (FIG. 3). The purified NK cells showed themselves to be the most effective population for killing ERK5-deficient cells. In contrast, the elimination of NK cells greatly reduced the cytotoxic activity of splenocytes towards ERK5-deficient cells. Interestingly, these cells turned out to be better targets for NK cells than other syngenic MHC-I-deficient cells, for example RMA-S or Yac-1 cells (FIG. 3B and data not shown). This very probably reflects the role of ERK5 in the activation of NF-(B, which would block apoptosis after engaging the death receptors (21). In summary, NK cells were responsible for the eradication of EL4-shERK5 cells in vitro (FIG. 3). These results were confirmed with another type of tumor cell, L1210 cells derived from a type B murine lymphoma (FIG. 10).

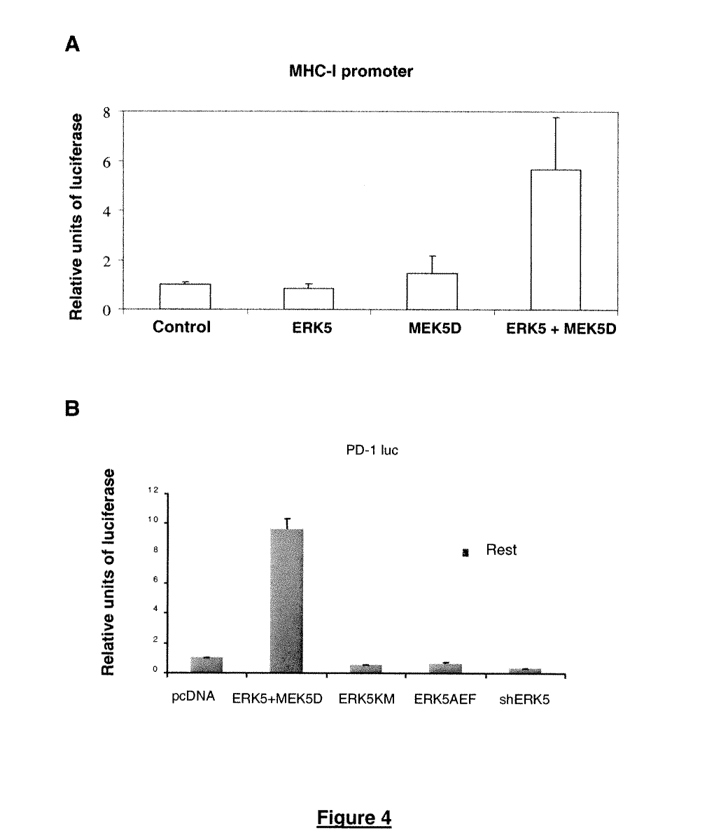

The inventors have shown that the expression of MHC-I regulated by ERK5 probably involves transcriptional regulation since over-expression of ERK5 coupled with a constitutivelyonally active MEK5 gene in Jurkat cells (human tumor cells) activated a reporter gene under the control of a MHC-I promoter derived from the class I gene (FIG. 4, (27)). In contrast, transfection of each protein separately did not activate the promoter.

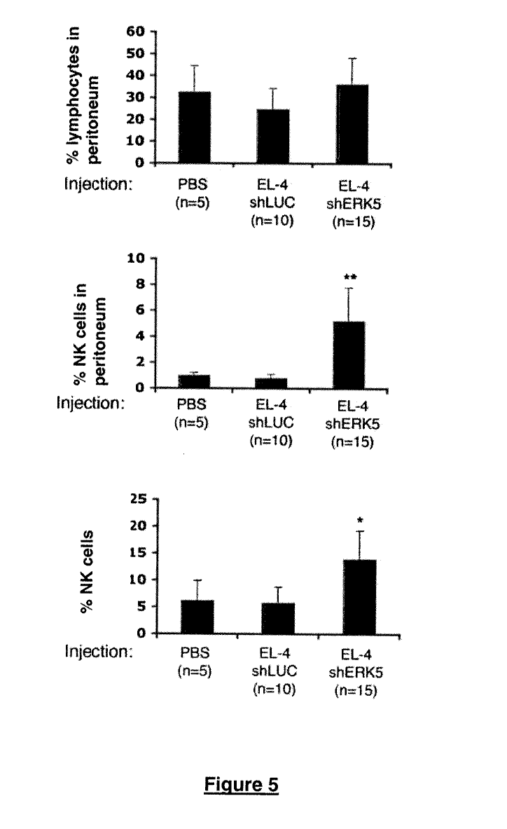

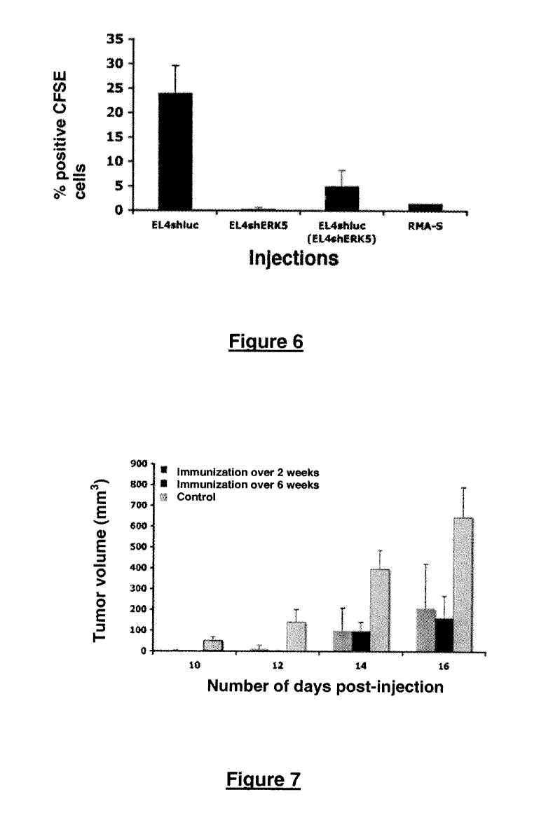

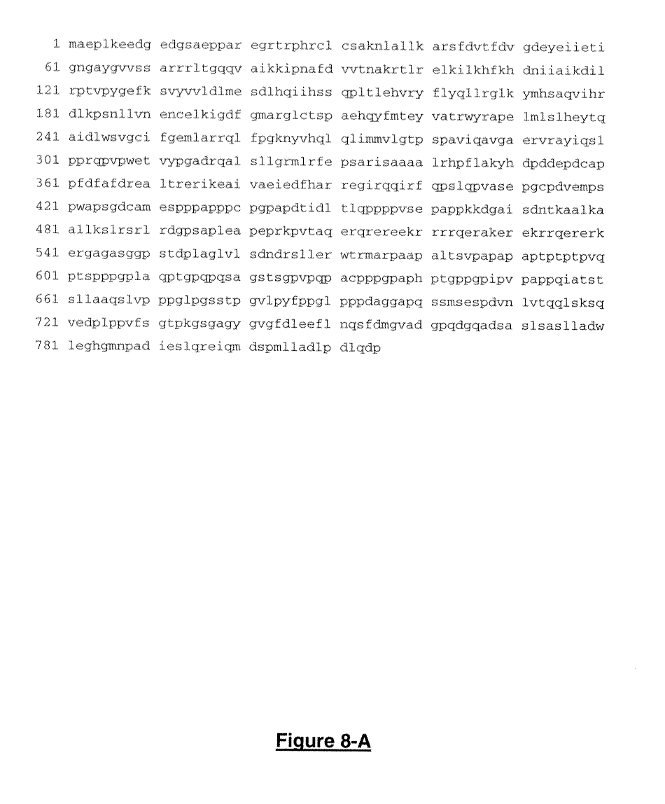

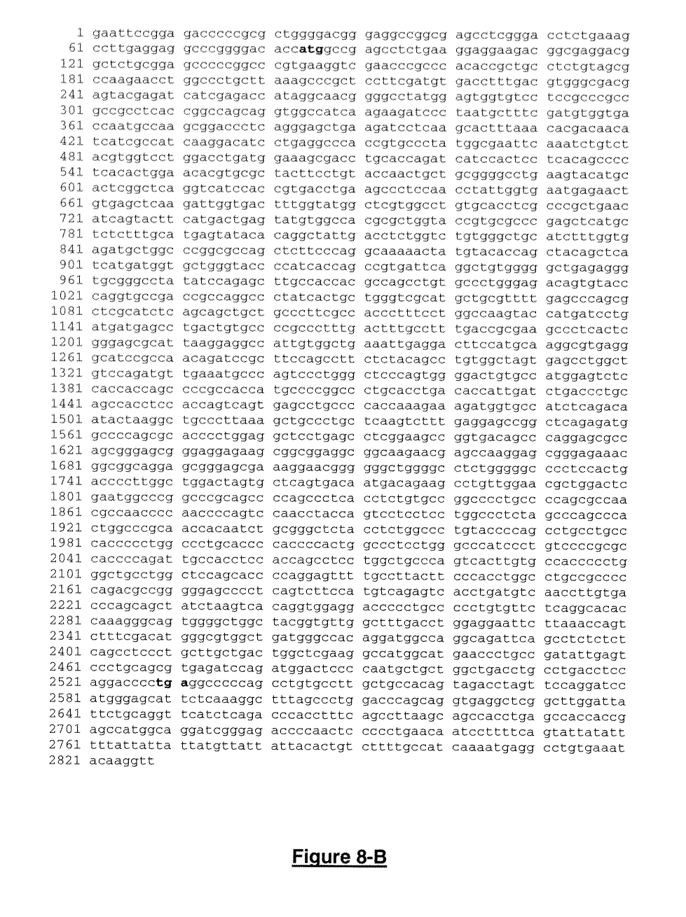

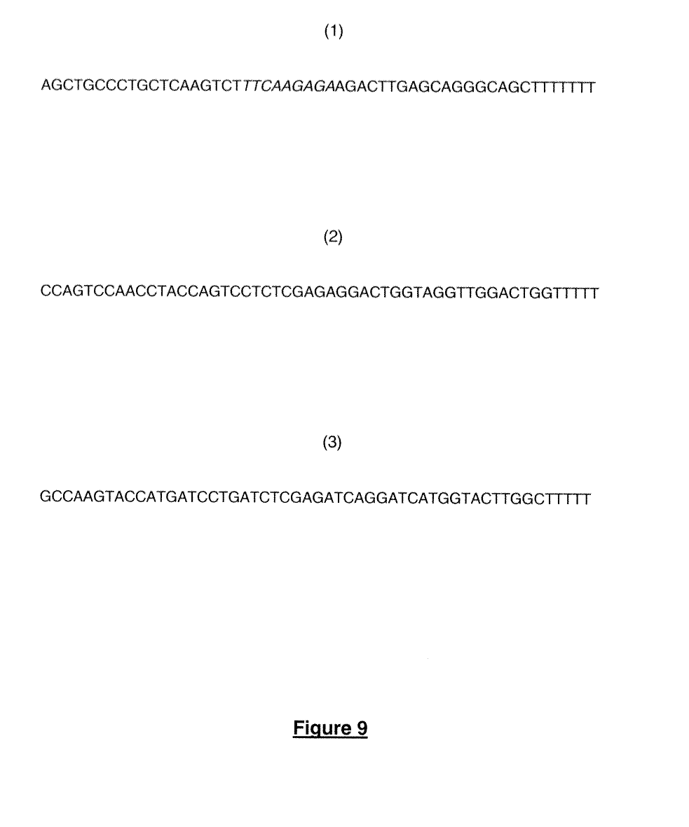

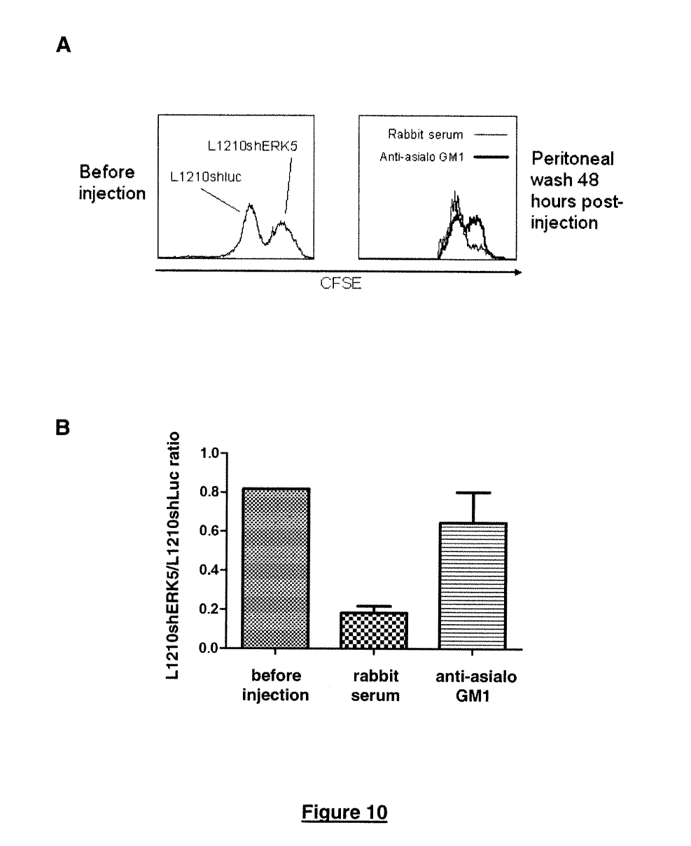

In vivo, NK cells may be recruited locally by inoculation of tumor cells, preferably those without appropriate MHC-I expression (22). For this reason, the inventors injected EL4-wt cells and shERK5 cells into the peritoneum of syngenic mice and three days later investigated the presence of NK cells (FIG. 5). The quantity of total lymphocytes did not increase notably following inoculation of EL4-shERK5 cells (FIG. 5A). In contrast, the number of NK cells was significantly higher in mice inoculated with shERK5 cells than in mice inoculated with control cells (FIG. 5B). Further, the percentage of NK cells increased in the population of peritoneal lymphocytes in mice having received injection of EL4-shERK5 cells (FIG. 5C). No changes were observed in these different populations when EL4-shLuc cells were injected.

The results obtained by the inventors strongly suggest that NK cells eliminated EL4-shERK5 cells in vivo. Recent results have brought to light the important role played by NK cells in tumoral immunosurveillance (30). The inventors investigated whether a prior injection, or vaccination, with EL4-shERK5 cells could affect tumor progression induced by EL4-wt cells. Co-injection of EL4-shERK5 and wt cells significantly reduced survival of wt cells in the peritoneum (FIG. 6), suggesting that EL4-shERK5 cells induce an immune response also directed against EL4-wt cells. In order to explore this hypothesis further, syngenic mice were vaccinated with EL4-shERK5 cells which resulted in a significant reduction in tumor development initiated by EL4-wt cells in 80% of mice and completely prevented the development of tumors in 20% of them (FIG. 7)

Finally, the inventors investigated whether tumor development could be inhibited in a more pertinent model in which T cell leukemias were induced by infecting newborn mice with the Moloney murine leukemia virus (M-MuLV) strain. T leukemic cells from the Yac-1 strain derived from a C57/B6 mouse infected with M-MuLV were injected into animals used as a control. Yac-1 cells, like EL4-shERK5 cells, do not express MHC-I on their surface and do not form tumors in vivo in our system. The newborn mice were inoculated intra-peritoneally with M-MuLV virus which induced T lymphomas with a latency of 3-4 months as described above (31).

60 days post-infection, the mice received injections of 150 000 Yac-1 cells or EL4-shERK5 cells every month for two months then every two weeks. The mice were examined regularly by palpation under anesthesia to detect organ enlargements, and bled to determine hematocrit. The results shown in Table 1 indicated that the mice were protected from leukemia by the EL4-shERK5 cells and to a less extent by the Yac-1 cells.

TABLE 1
Cells modified with shERK5 protect
mice from M-MuLV-induced leukemias.

| | | Number of mice surviving after | |
|---|---|---|---|
| Treatment | Total number of mice | five months | |
| None | 9 | 3 | (33.33%) |
| Yac-1 | 10 | 6 | (60%) |
| EL4-shERK5 | 7 | 7 | (100%) |

Newborn mice were infected with M-MuLV virus. Two months later, the mice were separated into three groups. The first group received no treatment. The second group received injections of Yac-1 cells and the third group received injections of EL4-shERK5 cells. The number of surviving mice five months post-infection is indicated.

### Example 2

The inventors carried out a second series of experiments with another type of murine tumor cell. L1210 cells derived from a type B murine lymphoma. The results are shown in Table 2 and FIGS. 10 to 18.

The results shown in Table 2 indicate that the L1210 cells transfected with an expression vector of a shRNA targeting ERK5 (“L1210-shERK5 cells”) do not induce tumors in vivo in the mouse.

TABLE 2
Two groups of mice received an injection of L1210 cells transfected
with an expression vector of a shRNA targeting ERK5 (“L1210-
shERK5 cells”) or L1210 cells transfected with the expression
vector shLuc used as a control (“L1210-shLuc cells”) respectively.
The tumor volume was measured after 8, 11 and 13 days.

| Sensitization | Volume (mm³) | Volume (mm³) | Volume (mm³) |
|---|---|---|---|
| vector at T0 | at T0 + 8 days | at T0 + 11 days | at T0 + 13 days |
| L1210-shLuc | 225 | 448.45 | 506.88 |
| L1210-shLuc | 62.21 | 31.9 | 302.69 |
| L1210-shLuc | 4 | 166.6 | 343.75 |
| L1210-shLuc | 231.04 | 530 | 671.55 |
| L1210-shERK5 | 4 | 15.75 | 13.5 |
| L1210-shERK5 | 4 | 32 | 62.5 |
| L1210-shERK5 | 0.5 | 4 | 4 |
| L1210-shERK5 | 4 | 4 | 4 |

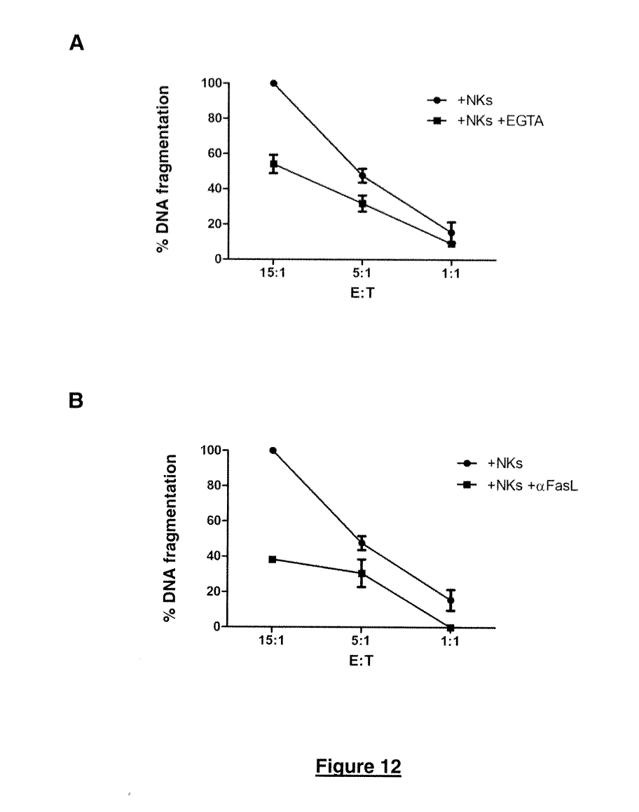

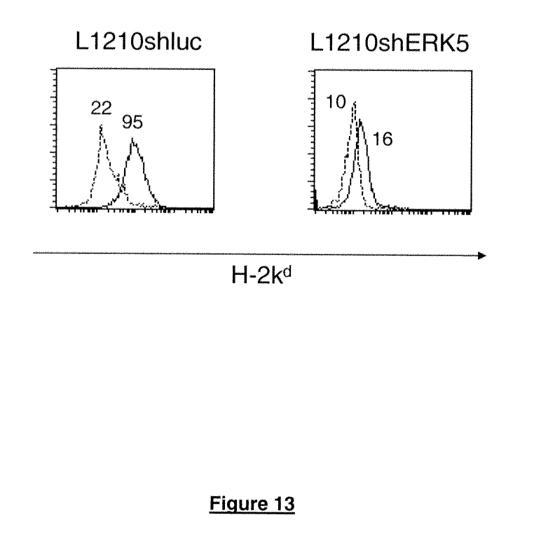

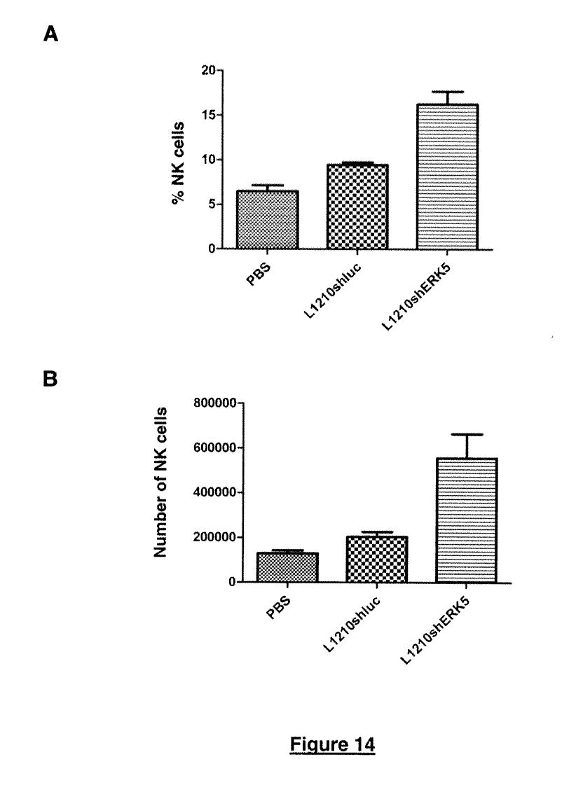

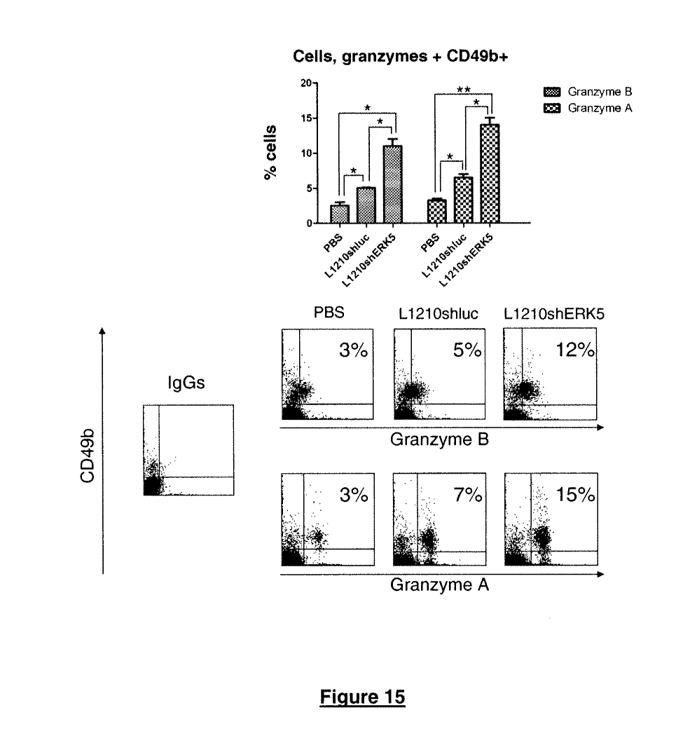

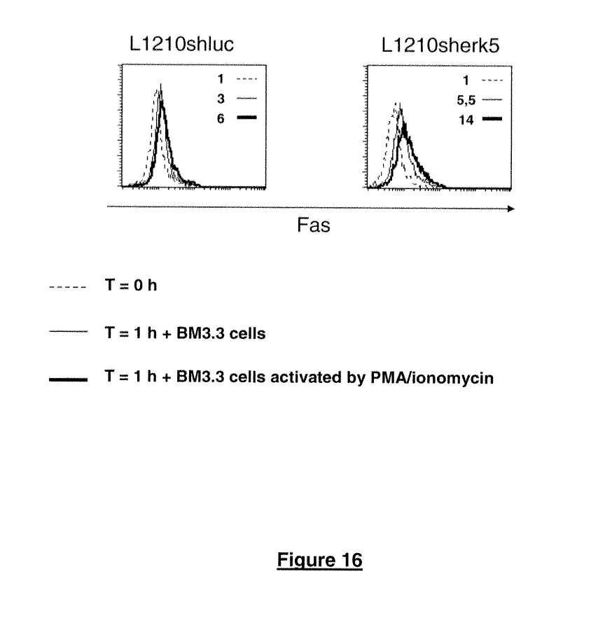

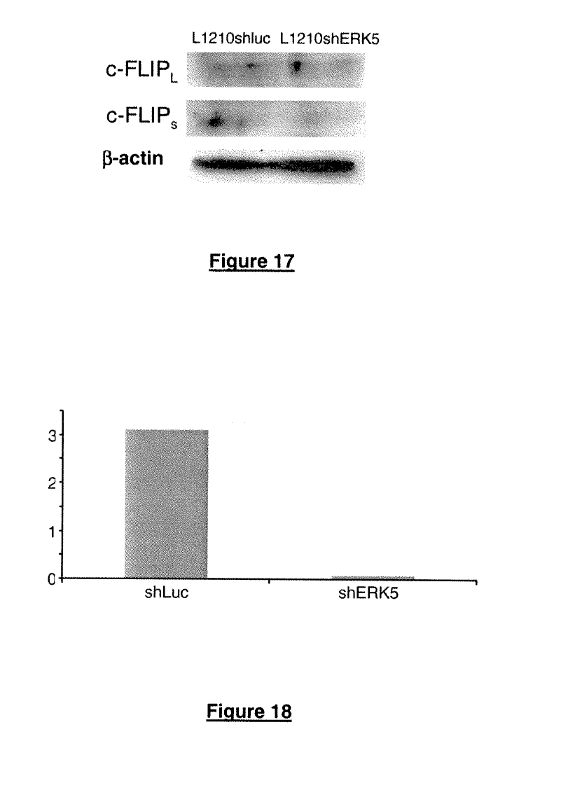

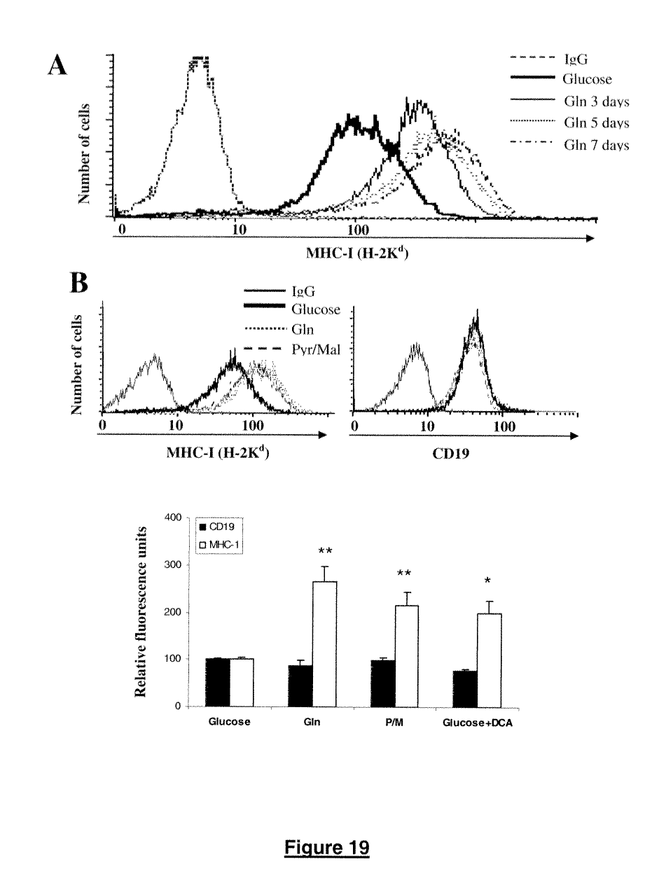

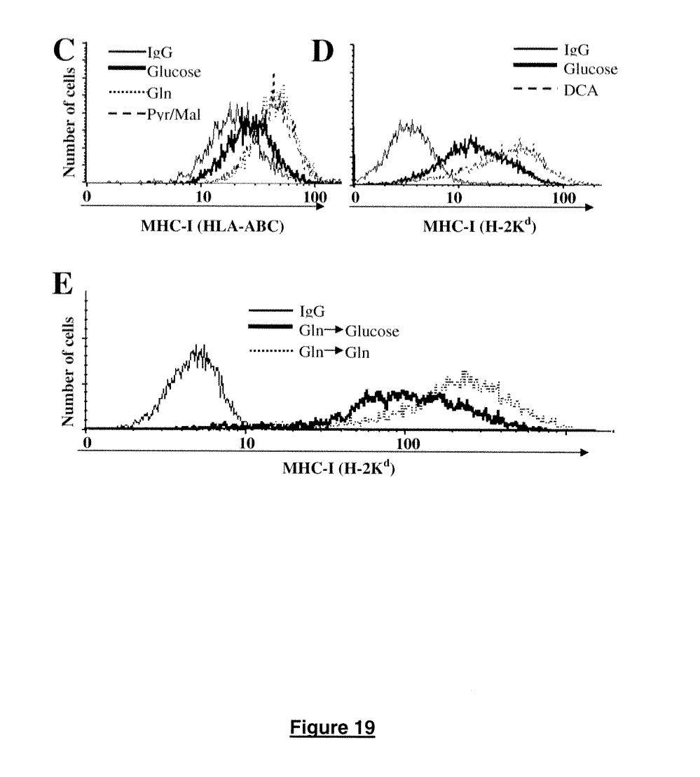

The inventors have also shown that L1210-shERK5 cells are eliminated from the peritoneum of untreated mice but not from the peritoneum of mice which received a prior treatment aimed at eliminating NK cells. L1210-shLuc cells, in contrast, were not eliminated from the peritoneum (FIG. 10). They have also shown that syngenic and allogenic NK cells both recognize L1210-shERK5 cells as foreign and eliminate them with similar efficiency (FIG. 11). The use of an anti-H2kd antibody (protein belonging to MHC-I) to evaluate by FACS the level of expression of MHC-I in L1210-shLuc or shERK5 cells showed that shERK5 cells lose the expression of MHC-I at the plasmid membrane (FIG. 13). The quantification of NK cells with an anti-CD49b antibody (protein expressed at the surface of NK cells) from mouse peritoneum cells recovered 48 hours following peritoneal injection of L1210-shLuc or shERK5 cells shows that the number as well as the proportion of NK cells in the peritoneum was much higher in the mice which had received shERK5 cells, thus showing that NK cells are recruited in the peritoneum by shERK5 cells (FIG. 14). The inventors then repeated this experiment by also quantifying, by FACS, the NK cells (CD49b+) expressing A and B granzymes, and thus showed that NK cells recruited by L1210-shERK5 cells are positive for granzymes and also as a consequence have an activated phenotype (FIG. 15). The inventors have also shown that incubation of L1210-shERK5 cells in the presence of syngenic NK cells and EGTA (which blocks cell death induced by granzymes) or an anti-FasL antibody (which blocks cells death induced by Fas), leads to a substantial reduction in the quantity of L1210-shERK5 cells eliminated by the NK cells, thereby demonstrating the importance of mechanisms involving granzymes and Fas in the elimination of shERK5 cells by NK cells (FIG. 12). The incubation of L1210-shLuc or shERK5 cells with the cytotoxic lymphocytes BM3.3 activated or not activated by a PMA/ionomycin treatment and labeled with an anti-Fas antibody proved that activated T lymphocytes (activated by the PMA/ionomycin treatment) induce a substantial increase in the level of expression of the death receptor Fas in L1210-shERK5 cells, which contributes to explaining the fact that these cells are highly sensitive to attacks by cytotoxic lymphocytes in general (FIG. 16). The inventors have also shown that shERK5 cells have reduced levels of the two isoforms, L and S, of the anti-apoptotic protein c-FLIP (FIG. 17). Finally, the inventors have shown that L1210-shERK5 cells present a larger reduction in the level of RNA of beta2-microglobulin, a protein which is essential to the stabilization of MHC-I molecules at the plasmid membrane level, which contributes to explaining why L1210-shERK5 cells express very small amounts of MHC-I protein at the plasmid membrane level (FIG. 18).

### Example 3

### Material and Methods

### 1) Reagents and Antibodies

The antibodies anti-H2 Kb-FITC, anti-H2 Kd-PE and anti-NK1.1-APC were from BD Pharmingen. The antibodies against ERK5 and β-actin were obtained from Cell Signalling Technology. Donkey anti-rabbit or sheep anti-mouse IgGs were obtained from Amersham. 5(6)-carboxyfluorescein diacetate, succinimidyl ester (CFSE) was obtained from Sigma-Aldrich.

### 2) Cell Culture

The EL4 T lymphocyte line cells, L1210 B lymphocyte line cells, Yac-1 cells, RMA-S, Jurkat and murine primary cells were cultivated in RPMI 1640-Glutamax (GIBCO) supplemented with 5% FBS and 50 μM 2-ME.

### 3) Plasmids

The expression vectors for ERK5, a constitutively active MEK5 mutant (S313D/T317D, termed MEK5DD) and β-galactosidase, were described above (21). pSiren-retroQ-puro (BD Biosciences) retroviral vectors for shERK5 and the shLuc control were described above (21). The MHC class I promoter construct used here derived from swine class I gene PD1 (27) and was a gift from Dr Dinah Singer (NCI/NIH).

### 4) Transient Transfection and Stable Cell Line Generation

Jurkat cells in logarithmic growth phase were transfected with the indicated amounts of plasmid by electroporation (21). In each experiment, cells were transfected with the same total amount of DNA by supplementing with empty vector. The cells were incubated for 10 min at ambient temperature with the DNA mixture and electroporated at 260 mV, 960 μF in 400 μl of RPMI 1640. Stable cell lines were generated as described previously (21). Briefly, cells were plated at 1.5×10⁶ cells/ml. One ml of a supernatant from 293T cells expressing the retroviral vector pSIREN for shERK5 or control plasmid was added after plating. Three days later, the cells were cultured with 2.5 μg/ml of puromycin (Sigma-Aldrich). After one week, surviving cells were isolated and kept on selection medium until used.

### 5) Reporter Assay

In all experiments, the cells were transfected with a β-galactosidase reporter plasmid (21). Transfected cells (1×10⁶) were harvested after 2 days and washed twice with PBS. The cells were lysed in 100 μl luciferase lysis buffer (Promega, Charbonniéres, France) and the luciferase assays (40 μl) were carried out according to the manufacturer's instructions (Promega, Charbonniéres, France) using a Berthold luminometer. To assay β-galactosidase, 40 μl of lysates was added to 200 μl of β-galactosidase buffer (50 mM of phosphate buffer, pH 7.4; ONPG 200 μg; 1 mM MgCl₂; 50 mM β-mercaptoethanol) and the absorbance was measured at 400 nm. The results are expressed as luciferase units normalized to the corresponding β-galactosidase activity. The level of expression of the transfected proteins was checked conventionally by immunoblot analysis.

### 6) CFSE Labeling and Peritoneal Clearance

Cells were labeled with CFSE as follows. One μl of a stock solution (5 mM) was diluted in 10 ml of PBS. Five million cells were washed once in PBS and re-suspended in 1 ml of the CFSE/PBS solution. After incubating for 2 min at ambient temperature, the cells were washed once with 10 ml of PBS and re-suspended at the indicated concentration. The indicated amounts of cells were injected intraperitoneally in 150 μl of PBS. The mice were sacrificed three days later and the peritoneal cells were collected, washed in PBS and analyzed on a FACSCalibur flow cytometer (Becton Dickinson).

### 7) Measure of Cytotoxic Activity

Spleen NK cells were isolated by positive selection using anti-DX5 magnetic beads (Miltenyi Biotec, Germany) according to the manufacturer's instructions. Briefly, viable single-cell suspensions were incubated (1 hour at 37° C.) on polystyrene tissue culture dishes. Non-adherent spleen cells (10⁷ cells) were incubated (15 minutes, 4° C.) with anti-DX5 magnetic beads, washed twice, and loaded (5×10⁷ cells) on a 25 LD column. The purity of the DX5⁺ cells collected was typically 98% (data not shown). The direct NK cell cytotoxic activity was assessed by labeling the target cells with Na₂⁵¹CrO₄ for one hour (EL4 cells) or with ³H-thymidine overnight (L1210 cells). Ten thousand target cells were mixed with the effector cells at the ratios indicated (for 4 hours at 37° C.) in 96-well V plates in a final volume of 200 microliters. The spontaneous ⁵¹Cr release (representative of cell lysis) was determined by incubating the target cells with medium alone. Maximum release was determined by adding 2.5% Triton X-100. For the experiments with ³H-thymidine (the % release of which is representative of DNA fragmentation), the cells were permeabilized with 25 μl of a solution containing 2% of Triton X-100, 80 mM of Tris/HCl, pH 7.5 and 8 mM of EDTA and incubating for 15 minutes at 37° C. The plates were centrifuged for 15 minutes at 400×g and 50 μl of supernatant was added to 2 ml of scintillation liquid (GE Healthcare). Spontaneous liberation was determined as the number of counts per minute (or cpm) in the absence of effector cells and total labeling was obtained by direct measurement of the target cells. The percentage specific lysis or DNA fragmentation was equal to:

[(sample cpm−spontaneous cpm)/(maximum cpm−spontaneous cpm)]×100

The spontaneous liberation was always less than 10% in the case of ⁵¹Cr (EL4 cells) and less than 20% with ³H-thymidine. All of the experiments were carried out three times.

### 8) Tumor Progression In Vivo

For subcutaneous tumor formation, EL4 cells were washed in PBS and re-suspended at 1.5×10⁶ cells/ml. A total of 2.5×10⁵ cells were injected subcutaneously into the flank of C57B/6 mice for pre-treatment with EL4-shERK5 cells and for inoculation of the EL4-shLuc cells. Tumor development was analyzed every 2 days. The tumors were measured, and the volume was calculated using the formula: V=length×(weight)²/2. For the M-MuLV-induced leukemia experiments, newborn mice were infected two days after birth with M-MuLV as previously described (28). Two months later, the mice were injected intraperitoneally with 2.5×10⁵ Yac-1 or EL4-shERK5 cells or PBS every 2 weeks. Leukemia progression was monitored by palpation and/or hematocrit measurement. All of the experiments involving animals were carried out according to the guidelines and regulations of the Centre Nationale de la Recherche Scientifique [National Center for Scientific Research].

### 9) Flow Cytometry

Peritoneal spleen cells (3×10⁶) or cell lines (2×10⁵) were stained for 20 minutes at ambient temperature with FITC- PE- or APC-conjugated antibodies in 200 μl PBS. Finally, the cells were washed and analyzed on a FACSCalibur flow cytometer (Becton Dickinson) using CellQuest software (Becton Dickinson).

### 10) Immunoblotting

Cells were washed with PBS and lysed in SDS-Laemmli buffer. The proteins were separated by 10% SDS-PAGE on minigels and processed for immunoblot analysis as previously described (21).

### 11) Statistical Analysis

The statistical analysis of the difference between the means of paired samples was carried out using the paired t-test. The results are given as the confidence interval (p). All of the experiments described in the figures were carried out at least three times with similar results.

### Example 4

### Changes in Tumor Cell Metabolism Underlines Tumor Cell Immune Escape

During the process of tumorigenesis cells are confronted to an adverse environment in two contexts: they must obtain nutrients for their rapid growth and they must escape the attack from the host immune system. Otto Warburg found in the 1920s ³⁷ that, even in the presence of ample oxygen, cancer cells prefer to metabolize glucose by anaerobic glycolysis (fermentation) that is less efficient for producing ATP than oxidative phosphorylation (OXPHOS, respiration). However, fermentation is much quicker than respiration on producing ATP and offers a selective advantage to rapidly growing tumor cells. Paul Ehrlich in 1909 was one of the first to conceive the idea that the immune system could repress a potentially “overwhelming frequency” of carcinomas. However this idea was not pursuit until the establishment of the existence of tumor-associated antigens (TAAs). In the 1960s, Macfarlane Burnet and Lewis Thomas proposed the hypothesis of “cancer immunosurveillance” ⁴² that is now commonly accepted ^{39, 43}. However, no relationship between cancer immunosurveillance and the Warburg effect has been described so far. Both processes occur early in tumor development suggesting that they could be linked. The appearance of clinically detectable tumors may be the result of the proliferation of highly selected tumor clones that develop sophisticated strategies to escape the immune response. Arguably, the most relevant is the total or selective loss of expression of the major histocompatibility complex class I (MHC-I). MHC-I mediates self-recognition and thus should present endogenously synthesized TAAs to CD8⁺ cytotoxic T lymphocytes (CTLs). Changes in MHC-I allows tumor cells to avoid CTLs and thereby the adaptive immune response ⁴⁰.

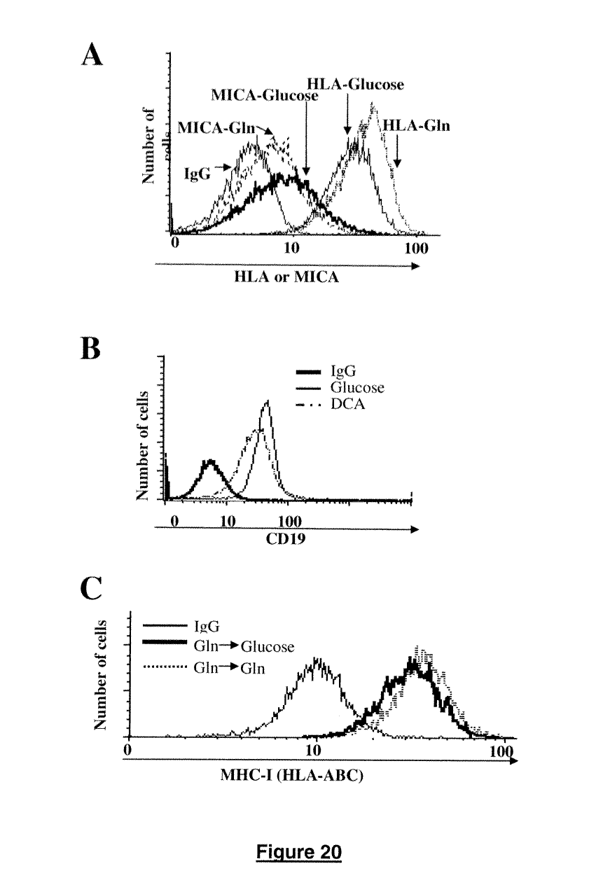

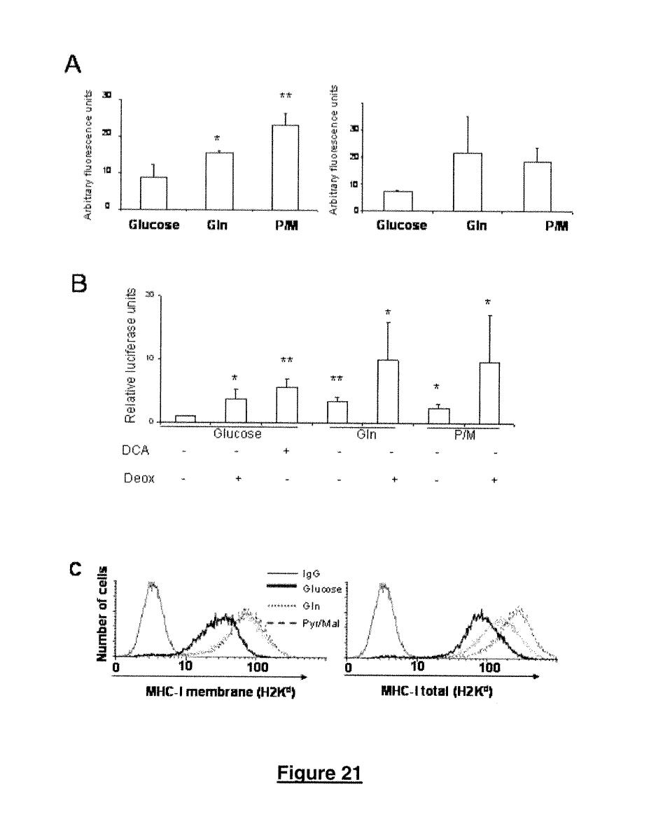

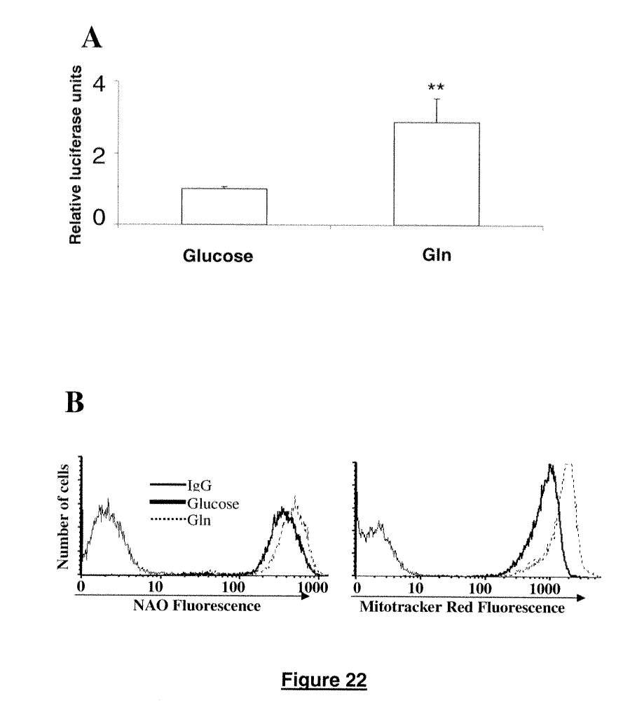

When glucose is no longer available cells are forced to use alternative energy substrates such as the oxidation of glutamine (Gln) that is present in most culture media. This process called glutaminolysis requires OXPHOS for ATP production ^{44, 45}. L1210 B leukemic cells growing in 25 mM glucose were changed to glucose-deprived medium supplemented with 10 mM galactose and 4 mM Gln (FIG. 19A). Galactose was added to Gln containing media to allow generation of nucleic acids through the pentose phosphate pathway ^{44, 45} MHC-I expression was analyzed by FACS analysis. Cells growing in Gln for three days show a 3-fold increase in MHC-I expression. Additional incubation for 2 or 4 days led to a small additional increase (FIG. 19A) and shorter incubations showed smaller increases (data not shown). Therefore, we mostly chose 3 days for subsequent studies. Cells growing in the presence of pyruvate and malate as respiratory substrates showed a similar increase (FIG. 19B, left panel). The expression of the B cell antigen CD19 (FIG. 19B, right panel) did not change in the different media. The lower panel of FIG. 19B showed the statistical significance of these results. Jurkat T cells also increased MHC-I expression (FIG. 19C), but not in the closely related MHC class I chain-related gene A (MICA; FIG. 20A). Dichloroacetate (DCA), a pyruvate dehydrogenase kinase (PDK1) inhibitor ⁴⁶, activates pyruvate dehygrogenase (PDH1) ⁴⁷ and forces pyruvate to enter the Kreb's cycle and therefore metabolism change to respiration even in high glucose media ⁴⁷. DCA increased MHC-I expression in the presence of 25 mM glucose (FIG. 19D). Again expression of CD19 was not altered (FIG. 19B and FIG. 20B). These data excluded that glucose-deprivation underlined our observations. Next, we examined if MHC-I up-regulation was reversible. L1210 or Jurkat cells that had up-regulated their MHC-I in Gln medium were placed on glucose- or Gln media. We did not observed reduced MHC-I levels 24 h later (data not shown). However, after 48 hours cells started decreasing their MHC-I (data not shown), reaching three days later basically the same levels that cells continuously growing in glucose (FIG. 19E and FIG. 20C). This meant that the metabolic status of tumor cells control expression of MHC-I at the plasma membrane. This expression is regulated at different levels including transcription ⁴⁰. Class I molecules consist of the light chain β2-microglobulin (β₂m; 12 kDa) and one heavy chain (45 kDa) encoded by several different genes within the MHC region: human leukocyte antigen (HLA) in humans and H-2 in mice. The molecular mechanisms underlying abnormal MHC class I expression in tumor cells include mutations or epigenetic changes in genes encoding β₂m, Class I Heavy Chain genes and components of the antigen presenting machinery (APM, ⁴⁰).

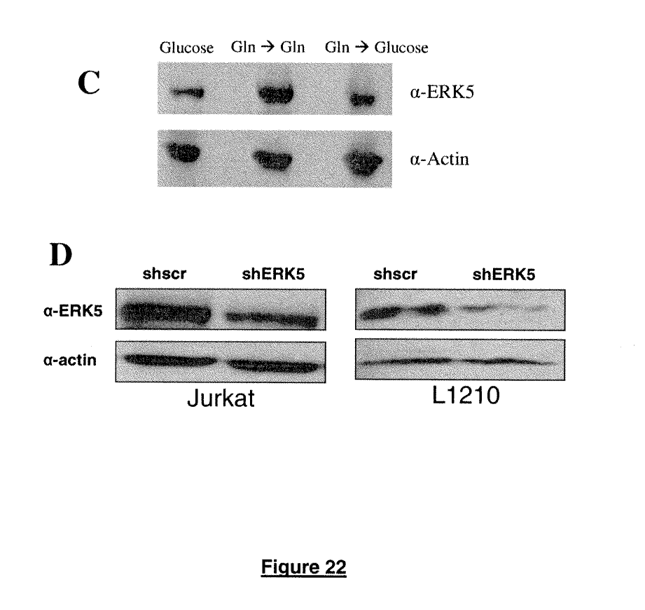

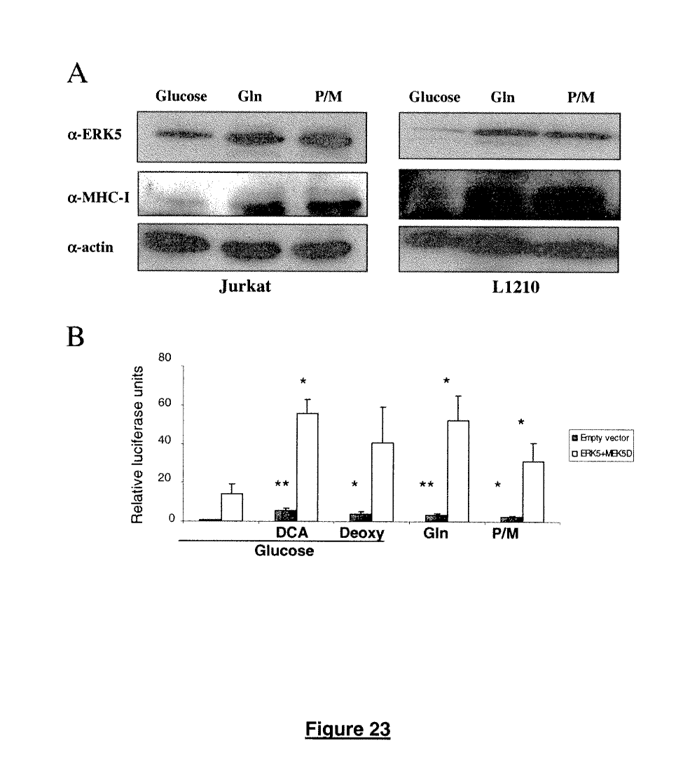

L1210 cells growing in OXPHOS media for 2 days showed higher mRNA expression of both β₂m and the heavy chain of the class I molecule H-2K^{d} (FIG. 21A). Moreover, Jurkat cells growing in those media increased expression of a transfected reporter gene controlled by the promoter of the MHC-I gene PD-1 (FIG. 21B) or the proximal promoter of the human HLA-A gene (FIG. 22A). In agreement with FIG. 19, this effect was not due to glucose deprivation because addition of DCA to glucose media also activated the PD-1 reporter (FIG. 21B). Our Gln media contained 5% of non-dialysed fetal bovine serum and therefore, around 0.5 mM glucose. To totally blocked glycolysis we used 2-deoxyglucose, which increased the expression of the PD-1 promoter in all media (FIG. 21B). The increased transcription of class I molecules correlated with higher total expression of MHC-I molecules in cells growing in OXPHOS media (FIGS. 21C and 23A). Therefore respiration induces de novo expression of MHC-I molecules and not only an increase in their translocation to the plasma membrane.

We have recently found that selective activation of the ERK5 pathway induces MHC-I expression in leukemic cells ⁴¹. FIG. 23A showed that leukemic cells growing in OXPHOS media increased ERK5 expression. In agreement with our previous studies ⁴¹, transfection of ERK5 and a constitutively active MEK5 mutant (MEK5D) activated the PD-1 gene reporter and strongly synergized with all tested OXPHOS conditions (FIG. 23B).

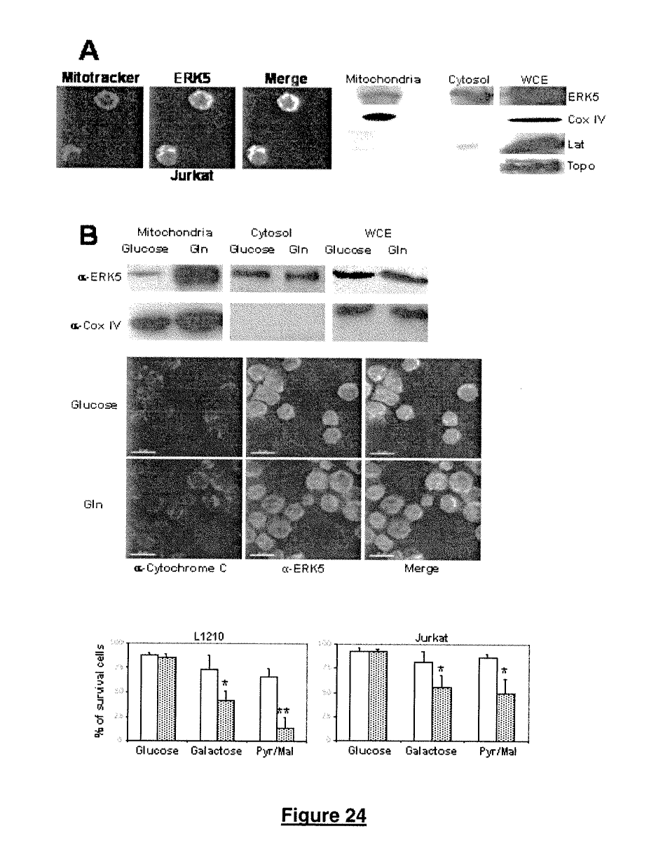

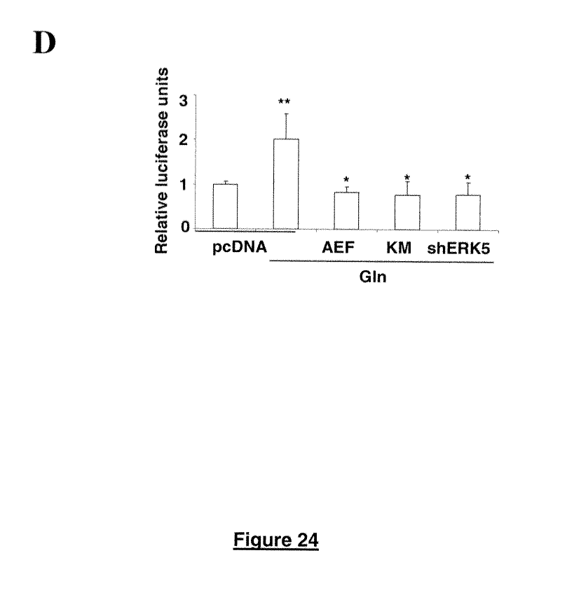

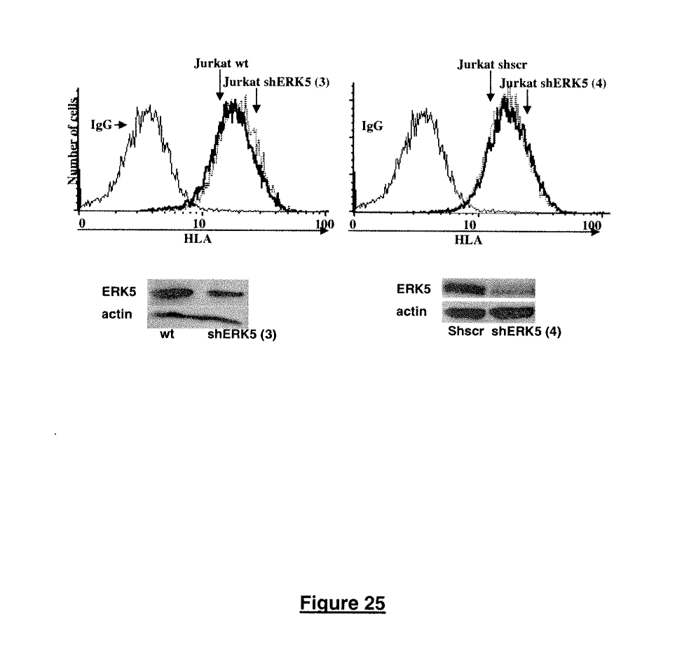

ERK5 subcellular localization depends on the cell type investigated and enrichment in cytosol or nucleus has been described. Immunofluorescence staining showed that ERK5 localized in the cytosol in Jurkat cells (FIG. 24A), including a compartment that stained positive with the mitochondrial marker mitotracker red (FIG. 24A). Quantitative measurement of protein-protein interaction showed that 20% of ERK5 colocalized with cytochrom C in Jurkat cells. We confirmed these results by biochemical studies where we found most ERK5 protein in the soluble cytosolic fraction but significant levels of ERK5 were also found in the mitochondrial fraction (FIG. 24A, right panels). This meant that whereas ERK5 localized in different compartments, mitochondria contained significant amounts of ERK5.

As expected ⁴⁵, L1210 cells growing in OXPHOS conditions increased the staining with Mitotracker Red or with nonyl acridine orange (NAO), that binds mitochondria independently of the mitochondrial membrane potential (FIG. 22). This correlated with higher ERK5 expression (FIG. 23) that, like MHC-I expression (FIG. 19E and FIG. 20C) was reversible (FIG. 22C). Under OXPHOS conditions most ERK5 protein colocalized with Cytochrome C (FIG. 24B, bottom panels) and was isolated in the mitochondrial fraction (FIG. 24B top panels). In fact, the largest fraction of de novo expressed ERK5 protein was found in mitochondria and not in cytosol (FIG. 24B top panels).

To investigate the role of ERK5 in cell metabolism, we generated leukemic cells stably expressing an shRNA for ERK5 (shERK5) or a scramble control (shscr). These cells showed approximately 50% reduction of ERK5 levels (FIG. 22D), similar to those described in MEF ERK5^{+/- 48}. Shscr, as well as wt, cells survived in OXPHOS media (FIG. 24C), although they proliferated slower than in glucose media (data not shown) as it has observed in other cell lines ⁴⁵. However, shERK5 cells showed a significant increased in cell death when forced performing respiration. DNA microarray analysis of shERK5-expressing Jurkat cells confirmed that these cells had mitochondrial defects (data not shown).

Tumor cells stably expressing shERK5 underwent apoptosis in OXPHOS media limiting their use for investigating the role of ERK5 in respiration-induce MHC-I expression. Transient inhibition of the ERK5 pathway by two dominant negative ERK5 construct (ERK5 KM and AEF) or by expression of shERK5 blocked galactose-induced activation of the PD-1 and HLA-A promoters (FIG. 24D).

During the process of tumorigenesis tumor cells are confronted to changes in their environment. Sometimes this can be adverse but cancer cells can also take advantage of these situations after selection of specific clones. Our results show that changing from respiration to fermentation decrease MHC-I expression and avoid the adaptive immune response in vivo. This will lead to natural selection of clones that by performing fermentation downregulate MHC-I expression, an observation largely observed in many tumors.

An important question is how general is this process. Our study shows that this happens in vitro in hematopoietic cells that show higher MHC-I levels than other cell types ⁴⁰ because of a specific enhanceosome ⁴⁹. Therefore this regulation could be restricted to hematopoietic cells like B or T leukemic cells. But, at least in vitro, cells in different metabolic state could not show this regulation, e.g. cells that already use their mitochondria would show a lower effect after glucose deprivation. Moreover, because a high percentage of cell lines in culture show intrinsic deficiencies in MHC-I expression e.g. mutations in heavy an light chains of class I molecules ⁴⁰, it is possible that this phenomenon could not be observed in some cell lines.

An interesting issue is how ERK5 regulates metabolism. Our DNA microarray assay using shERK5-expressing cells show strong alteration in metabolic pathways that could be mediated by the selective, but partial, ERK5 localization in the mitochondria. ERK5 regulates genes associated with hypoxia via regulation by HIF-1 ⁵⁰ however ERK5 regulates these genes in normoxia, not in hypoxia. Moreover, almost 30% of oral squamous cell carcinomas (OSCCs) show increased expression of ERK5 ⁵¹. These carcinomas show significant downregulation of electron transport chain genes. Taken together all these results suggests that ERK5 controls expression of different metabolic processes and not only those regulated by HIF. Therefore, our results uncover why minimum ERK5 levels are essential for cell survival ^{48, 21, 10, 52}.

In addition to the new insights in the field of cancer, our results could also explain why embryonic cells, which show a more anaerobic glycolytic metabolism than mature cells, lack MHC-I expression ⁵³. In fact, our very preliminary results show that mouse embryonic fibroblasts (MEFs) growing in galactose medium increased MHC-I expression (data not shown). This would mean that tumor cells undergo to an embryonic-like metabolism ^{37, 47} and acquire expression of embryonic-like antigens e.g. low MHC-I expression.

This data supported the notion that ERK5 played an important role in cell metabolism that would explain why mammal cells needed a minimum level of ERK5 protein to survive ^{48, 21, 10, 52}.

Some of the earliest studies of cancer noted metabolic differences between normal tissue and tumors ¹. For example, tumor cells maintain a high glycolytic rate even in conditions of adequate oxygen supply, i.e. the Warburg effect. Therefore, tumor cells produce ATP primarily by fermentation instead of respiration. Fermentation generates less ATP but is quicker than respiration and offers a selective advantage to tumors, possibly when resources are limiting ⁴⁷. The Warburg effect is sufficiently ubiquitous in human cancers to permit tumor detection by positron emission tomography (PET). Analysis of the signaling pathways and molecular actors involved in metabolic remodeling revealed a central role for the transcription regulator hypoxia inducible factor 1□ (HIF1□), which induces the expression of particular (rapid) isoforms of most glycolytic enzymes ⁵⁴. HIF1□ inhibits pyruvate dehydrogenase (PDH) by upregulation of its kinase (PDK1; ^{55, 56}). This lowers the conversion of pyruvate to acetyl-CoA and ultimately to reduced ATP production by the respiratory chain, thereby enhancing the Warburg effect and cancer cell growth. Interestingly, selective activation of extracellular-regulated kinase 5 (ERK5) leads to strong upregulation of HIF1□-regulated genes ⁵⁰. ERK5 does not directly activate HIF1□ and is not activated by hypoxia. This suggests that ERK5 is essential for regulation of HIF1□-regulated genes under normoxia.

ERK5 ^{2, 3}, which is present in primary and leukemic cells ⁴, shares the TEY activation motif with other ERKs, while its other structural features are unique, such as the large regulatory C terminus that controls its nucleo-cytoplasmic shuttling ⁵. The essential role for ERK5 is underline by the fact that mouse embryonic fibroblasts (MEF) lacking ERK5 expression undergo spontaneous apoptosis ⁴⁸. Moreover, blocking the expression of one ERK5 allele significantly increases spontaneous apoptosis ⁴⁸. Our results ^{21, 52} show that downregulation of ERK5 levels by expression of a small hairpin RNA (shERK5) induces spontaneous apoptosis in leukemic cell lines. Thus, a certain level of ERK5, and not just its activation, is critical for cell survival.

Methods Summary

The leukemic T cell line Jurkat and the murine leukemic B L1210 cell line were grown in RPMI 1640—Glutamax (GIBCO) supplemented with 6% FBS. In certain experiments cells were incubated in RPMI 1640 (GIBCO 11879) that contains 2 mM glutamine, but no glucose. This media was supplemented with 25 mM glucose (glucose-medium), 2 mM glutamine (Gln-medium) or 12.5 mM each pyruvate plus malate (pyruvate-medium).

The MHC class I promoter constructs are derived from the swine class I gene PD1 ⁴⁹ and the human HLA-A gene ⁵⁷ have been described.

For Flow Cytometry, cell lines (1×10⁵) were stained for 20 minutes at RT with indicated FITC-, PE- or APC-conjugated antibodies in 200 □l PBS. Cells were washed and analysed on FACSCalibur flow cytometer Becton Dickinson using CellQuestPro software (Becton Dickinson). For total MHC-I expression in permeabilized cells, L1210 cells growing in different media were incubated for 15 min with anti-H2K^{d}-PE, washed with PBS and fixed with 2% PFA for 15 min. Next, they were permeabilized with 0.5% saponin, and incubated with anti-H2K^{d}-PE (total MHC-I expression) or control IgG-PE (membrane MHC-I expression). After PBS washing cells were analyzed by FACs.

Immunofluorescence confocal images were acquired using a Zeiss LSM 510 inverted laser-scanning microscope equipped with an external argon laser, using a Zeiss fluor 63× objective. Analyses were done with Imaris software. The extent of colocalization of two labels was measured using the “Colocalization” module of Imaris 5.0.2., 64-bit version (Bitplane AG, Saint Paul, Minn., USA, www.bitplane.com). This program analyzes stacks of confocal sections acquired in two channels. Each confocal section consists of an array of square elements called pixels. A voxel is defined from a pixel as a prism in which the base is the pixel and the height is the thickness of the confocal section. Imaris colocalization analyzes the entire confocal stack by measuring the intensity of each label in each voxel. The program uses an iterative procedure ⁵⁸ to determine an intensity threshold (in the 0-255 scale of pixel intensity) for each of the two labels. Voxels with intensities above this threshold are considered to be above the background. A voxel is defined as having colocalization when the intensities of both labels are above their respective thresholds. To avoid investigator bias in setting the thresholds, the program has an automatic thresholding feature ⁵⁸.

### REFERENCES

1. Bard, D., H. C. Maguire, Jr., P. McCue, and M. J. Mastrangelo. 1990. Treatment of metastatic melanoma with an autologous tumor-cell vaccine: clinical and immunologic results in 64 patients. J Clin Oncol 8:1858-1867.
2. Lee, J. D., R. J. Ulevitch, and J. Han. 1995. Primary structure of BMK1: a new mammalian map kinase. Biochem Biophys Res Commun 213:715-724.
3. Zhou, G., Z. Q. Bao, and J, E. Dixon. 1995, Components of a new human protein kinase signal transduction pathway. J Biol Chem 270:12665-12669.
4. Garaude, J., S. Kaminski, S. Cherni, R. A. Hipskind, and M. Villalba. 2005. The Role of ERK5 in T-Cell Signalling. Scand J Immunol 62:515-520.
5. Buschbeck, M., and A. Ullrich. 2005. The Unique C-terminal Tail of the Mitogen-activated Protein Kinase ERK5 Regulates Its Activation and Nuclear Shuttling. J Biol Chem 280:2659-2667.
6. Esparis-Ogando, A., E. Diaz-Rodriguez, J. C. Montero, L. Yuste, P. Crespo, and A. Pandiella. 2002. Erk5 participates in neuregulin signal transduction and is constitutively active in breast cancer cells overexpressing ErbB2. Mol Cell Biol 22:270-285.
7. English, J. M., G. Pearson, T. Hockenberry, L. Shivakumar, M, A. White, and M. H, Cobb. 1999. Contribution of the ERK5/MEK5 pathway to Ras/Raf signaling and growth control. Journal of Biological Chemistry 274:31588-31592.
8. Mulloy, R., S. Salinas, A. Philips, and R. A. Hipskind. 2003. Activation of cyclin D1 expression by the ERK5 cascade. Oncogene 22:5387-5398.
9. Linnerth, N. M., M. Baldwin, C. Campbell, M. Brown, H. McGowan, and R. A. Moorehead. 2005. IGF-II induces CREB phosphorylation and cell survival in human lung cancer cells. Oncogene 24:7310-7319.
10. Buschbeck, M., S. Hofbauer, L. D. Croce, G. Keri, and A. Ullrich. 2005. Abl-kinase-sensitive levels of ERK5 and its intrinsic basal activity contribute to leukaemia cell survival. EMBO Rep 6:63-69.
11. Carvajal-Vergara, X., S. Tabera, J. C. Montero, A. Esparis-Ogando, R. Lopez-Perez, G. Mateo, N. Gutierrez, M. Parmo-Cabanas, J. Teixido, J. F. San Miguel, and A. Pandiella. 2005. Multifunctional role of Erk5 in multiple myeloma. Blood 105:4492-4499.
12. Mehta, P. B., B. L. Jenkins, L. McCarthy, L. Thilak, C. N. Robson, D. E. Neal, and H. Y. Leung. 2003. MEK5 overexpression is associated with metastatic prostate cancer, and stimulates proliferation, MMP-9 expression and invasion. Oncogene 22:1381-1389.
13. Weldon, C. B., A. B. Scandurro, K. W. Rolfe, J. L. Clayton, S. Elliott, N. N. Butler, L. I. Melnik, J. Alam, J. A. McLachlan, B. M. Jaffe, B. S. Beckman, and M. E. Burow. 2002. Identification of mitogen-activated protein kinase as a chemoresistant pathway in MCF-7 cells by using gene expression microarray. Surgery 132:293-301.
14. Nagel, S., C. Burek, L. Venturini, M. Scherr, H. Quentmeier, C. Meyer, A. Rosenwald, H. G. Drexler, and R. A. Macleod. 2006. Comprehensive analysis of homeobox genes in hodgkin lymphoma cell lines identifies dysregulated expression of HOXB9 mediated via ERK5 signaling and BMI1. Blood.
15. Sohn, S. J., B. K. Sarvis, D. Cado, and A. Winoto. 2002. ERK5 MAPK regulates embryonic angiogenesis and acts as a hypoxia-sensitive repressor of vascular endothelial growth factor expression. J Biol Chem 277:43344-43351.
16. Regan, C. P., W. Li, D. M. Boucher, S. Spatz, M. S. Su, and K. Kuida. 2002. Erk5 null mice display multiple extraembryonic vascular and embryonic cardiovascular defects. Proc Natl Acad Sci USA 99:9248-9253.
17. Wang, X., A. J. Merritt, J. Seyfried, C. Guo, E. S. Papadakis, K. G. Finegan, M. Kayahara, J. Dixon, R. P. Boot-Handford, E. J. Cartwright, U. Mayer, and C. Tournier. 2005. Targeted deletion of MEK5 causes early embryonic death and defects in the extracellular signal-regulated kinase 5/myocyte enhancer factor 2 cell survival pathway. Mol Cell Biol 25:336-345.
18. Hayashi, M., S. W. Kim, K. Imanaka-Yoshida, T. Yoshida, E. D. Abel, B. Eliceiri, Y. Yang, R. J. Ulevitch, and J. D. Lee. 2004. Targeted deletion of BMK1/ERK5 in adult mice perturbs vascular integrity and leads to endothelial failure. J Clin Invest 113:1138-1148.
19. Hayashi, M., C. Fearns, B. Eliceiri, Y. Yang, and J. D. Lee. 2005. Big mitogen-activated protein kinase 1/extracellular signal-regulated kinase 5 signaling pathway is essential for tumor-associated angiogenesis. Cancer Res 65:7699-7706.
20. Wang, X., K. G. Finegan, A. C. Robinson, L. Knowles, R. Khosravi-Far, K. A. Hinchliffe, R. P. Boot-Handford, and C. Tournier. 2006. Activation of extracellular signal-regulated protein kinase 5 downregulates FasL upon osmotic stress. Cell Death Differ.
21. Garaude, J., S. Cherni, S. Kaminski, E. Delepine, C. Chable-Bessia, M. Benkirane, J. Borges, A. Pandiella, M. A. Iniguez, M. Fresno, R. A. Hipskind, and M. Villalba. 2006. ERK5 activates NF-kappaB in leukemic T cells and is essential for their growth in vivo. J Immunol 177:7607-7617.
22. Glas, R., L. Franksson, C. Une, M. L. Eloranta, C. Ohlen, A. Orn, and K. Karre. 2000. Recruitment and activation of natural killer (NK) cells in vivo determined by the target cell phenotype. An adaptive component of NK cell-mediated responses. J Exp Med 191:129-138.
23. Hallett, W. H., and W. J. Murphy. 2006. Positive and negative regulation of Natural Killer cells: Therapeutic implications. Semin Cancer Biol 16:367-382.
24. Wodnar-Filipowicz, A., and C. P. Kalberer. 2006. Function of natural killer cells in immune defense against human leukaemia. Swiss Med Wkly 136:359-364.
25. Passweg, J. R., A. Tichelli, S. Meyer-Monard, D. Heim, M. Stern, T. Kuhne, G. Favre, and A. Gratwohl. 2004. Purified donor NK-lymphocyte infusion to consolidate engraftment after haploidentical stem cell transplantation. Leukemia 18:1835-1838.
26. Miller, J. S., Y. Soignier, A. Panoskaltsis-Mortari, S. A. McNearney, G. H. Yun, S. K. Fautsch, D. McKenna, C. Le, T. E. Defor, L. J. Burns, P. J. Orchard, B. R. Blazer, J. E. Wagner, A. Slungaard, D. J. Weisdorf, I. J. Okazaki, and P. B. McGlave. 2005. Successful adoptive transfer and in vivo expansion of human haploidentical NK cells in patients with cancer. Blood 105:3051-3057.
27. Howcroft, T. K., J. D. Weissman, A. Gegonne, and D. S. Singer. 2005. A T lymphocyte-specific transcription complex containing RUNX1 activates MHC class I expression. J Immunol 174:2106-2115.
28. Audit, M., J. Dejardin, B. Hohl, C. Sidobre, T. J. Hope, M. Mougel, and M. Sitbon. 1999. Introduction of a cis-acting mutation in the capsid-coding gene of moloney murine leukemia virus extends its leukemogenic properties. J Virol 73:10472-10479.
29. Gays, F., M. Unnikrishnan, S. Shrestha, K. P. Fraser, A. R. Brown, C. M. Tristram, Z. M. Chrzanowska-Lightowlers, and C. G. Brooks. 2000. The mouse tumor cell lines EL4 and RMA display mosaic expression of NK-related and certain other surface molecules and appear to have a common origin. J Immunol 164:5094-5102.
30. Suck, G. 2006. Novel approaches using natural killer cells in cancer therapy. Semin Cancer Biol 16:412-418.
31. Fan, H. 1997. Leukemogenesis by Moloney murine leukemia virus: a multistep process. Trends Microbiol 5:74-82.
32. Kawabata, H., A. Niwa, S. Tsuji-Kawahara, H. Uenishi, N. Iwanami, H. Matsukuma, H. Abe, N. Tabata, H. Matsumura, and M. Miyazawa. 2006. Peptide-induced immune protection of CD8+ T cell-deficient mice against Friend retrovirus-induced disease. Int Immunol 18:183-198.
33. Messer, R. J., U. Dittmer, K. E. Peterson, and K. J. Hasenkrug. 2004. Essential role for virus-neutralizing antibodies in sterilizing immunity against Friend retrovirus infection. Proc Natl Acad Sci USA 101:12260-12265.
34. Reuter, T., M. Heldmann, S. Schimmer, K. Schepers, and U. Dittmer. 2004. Protection of mice against Friend retrovirus infection by vaccination with antigen-loaded, spleen-derived dendritic cells. Vaccine 22:2686-2689.
35. Akao, Y., Nakagawa Y., Kitade Y., Kinoshita T., and Naoe T. 2007. Downregulation of microRNAs-143 and -145 in B-cell malignancies. Cancer Sci 98:1914-1920.
36. Radu D. R., Lai C-Y., Jeftinija K., Rowe E. W., Jeftinja S, and Lin V. S-Y. 2004. A Polyamidoamine Dendrimer-Capped Mesoporous Silica Nonosphere-Based Gene Transfection Reagent. J. Am. Chem. Soc. 126:13216-13217.
37. Warburg, O. On respiratory impairment in cancer cells. Science 124, 269-70 (1956).
38. Gogvadze, V., Orrenius, S. & Zhivotovsky, B. Mitochondria in cancer cells: what is so special about them? Trends Cell Biol 18, 165-73 (2008).
39. Dunn, G. P., Bruce, A. T., Ikeda, H., Old, L. J. & Schreiber, R. D. Cancer immunoediting: from immunosurveillance to tumor escape. Nat Immunol 3, 991-8 (2002).
40. Aptsiauri, N. et al. MHC class I antigens and immune surveillance in transformed cells. Int Rev Cytol 256, 139-89 (2007).
41. Charni, S. et al. ERK5 Knockdown generates mouse leukemia cells with low MHC class i levels that activate NK cells and block tumorigenesis. J Immunol 182, 3398-405 (2009).
42. Burnet, F. M. The concept of immunological surveillance. Prog Exp Tumor Res 13, 1-27 (1970).
43. Dunn, G. P., Koebel, C. M. & Schreiber, R. D. Interferons, immunity and cancer immunoediting. Nat Rev Immunol 6, 836-48 (2006).
44. Reitzer, L. J., Wice, B. M. & Kennell, D. Evidence that glutamine, not sugar, is the major energy source for cultured HeLa cells. J Biol Chem 254, 2669-76 (1979).
45. Rossignol, R. et al. Energy substrate modulates mitochondrial structure and oxidative capacity in cancer cells. Cancer Res 64, 985-93 (2004).
46. Whitehouse, S., Cooper, R. H. & Randle, P. J. Mechanism of activation of pyruvate dehydrogenase by dichloroacetate and other halogenated carboxylic acids. Biochem J 141, 761-74 (1974).
47. Bartrons, R. & Caro, J. Hypoxia, glucose metabolism and the Warburg's effect. J Bioenerg Biomembr 39, 223-9 (2007).
48. Wang, X. at al. Activation of extracellular signal-regulated protein kinase 5 downregulates FasL upon osmotic stress. Cell Death Differ (2006).
49, Howcroft, T. K., Weissman, J. D., Gegonne, A. & Singer, D. S. A T lymphocyte-specific transcription complex containing RUNX1 activates MHC class I expression. J Immunol 174, 2106-15 (2005).
50. Schweppe, R. E., Cheung, T. H. & Ahn, N. G. Global gene expression analysis of ERK5 and ERK1/2 signaling reveals a role for HIF-1 in ERK5-mediated responses. J Biol Chem 281, 20993-1003 (2006).
51. Sticht, C. et al. Activation of MAP kinase signaling through ERK5 but not ERK1 expression is associated with lymph node metastases in oral squamous cell carcinoma (OSCC). Neoplasia 10, 462-70 (2008).
52. Garaude, J. et al. SUMOylation regulates the transcriptional activity of JunB in T lymphocytes. J Immunol 180, 5983-90 (2008).
53. van den Elsen, P. J., Holling, T. M., van der Stoep, N. & Boss, J. M. DNA methylation and expression of major histocompatibility complex class I and class II transactivator genes in human developmental tumor cells and in T cell malignancies. Clin Immunol 109, 46-52 (2003).
54. Weidemann, A. & Johnson, R. S. Biology of HIF-1alpha. Cell Death Differ 15, 621-7 (2008).
55. Kim, J. W., Tchernyshyov, I., Semenza, G. L. & Dang, C. V. HIF-1-mediated expression of pyruvate dehydrogenase kinase: a metabolic switch required for cellular adaptation to hypoxia. Cell Metab 3, 177-85 (2006).
56. Papandreou, I., Cairns, R. A., Fontana, L., Lim, A. L. & Denko, N. C. HIF-1 mediates adaptation to hypoxia by actively downregulating mitochondrial oxygen consumption. Cell Metab 3, 187-97 (2006).
57. Gobin, S. J. & van den Elsen, P. J. Transcriptional regulation of the MHC class Ib genes HLA-E, HLA-F, and HLA-G. Hum Immunol 61, 1102-7 (2000).
58. Costes, S. V. at al. Automatic and quantitative measurement of protein-protein colocalization in live cells. Biophys J 86, 3993-4003 (2004).

1. A living animal tumor cell presenting a negative MHC-I phenotype useful as a medicament or vaccine.

2. The tumor cell according to claim 1, wherein over 40% of said tumor cells (T) are lysed in vitro after 4 hours in presence of syngenic non activated NK cells (E) at an E:T ratio of 5:1.

3. A living animal tumor cell, isolated or in culture, presenting a negative ERK5 phenotype and a negative MHC-I phenotype useful as a medicament or vaccine.

4. The tumor cell according to claim 3, comprising an agent of exogenous origin which is capable under appropriate culture conditions of reducing:
the level of expression or the quantity of endogenous ERK5 protein; and optionally,
the overall level of expression or the overall quantity of MHC-I proteins present at the cell surface.

5. The tumor cell according to claim 4, comprising a second agent of exogenous origin which is capable under appropriate culture conditions of reducing the overall level of expression or the overall quantity of MHC-I proteins present at the cell surface.

6. The tumor cell according to claim 3 having:
a reduction of 25% to 90% in the level of expression or the quantity of ERK5 protein present in the cell compared with its level of expression or its quantity in the absence of said exogenous agent under identical culture conditions; and
a reduction of 50% to 100% in the overall level of expression or the overall quantity of MHC-I proteins present at the cell surface compared with their overall level of expression or their overall quantity at the cell surface in the absence of said exogenous agent under identical culture conditions.

7. The tumor cell according to claim 3, wherein the negative ERK5 phenotype is caused at least in part by a post-transcriptional mechanism.

8. The tumor cell according to claim 7, wherein said cell comprises a nucleic acid molecule of exogenous origin comprising a sequence of 15 to 25 nucleotide residues having a degree of homology of at least 85% with a portion of the nucleotide sequence of the gene or the cDNA encoding the human ERK5 protein represented by the sequence SEQ ID NO: 2 or one of its animal equivalents.

9. The tumor cell according to claim 8, wherein said nucleic acid molecule of exogenous origin is:
a molecule of the miRNA, siRNA, antisense RNA, sense RNA or ribozyme type; or
a molecule of DNA comprising a sequence the transcription of which generates a molecule of RNA of the shRNA, antisense RNA, sense RNA, miRNA, siRNA or ribozyme type, under the control of a promoter which is active in the cell.

10. The tumor cell according to claim 9, wherein said post-transcriptional mechanism is a RNA interference mechanism.

11. The tumor cell according to claim 10, wherein said molecule of nucleic acid of exogenous origin comprises a molecule of DNA comprising one of sequences SEQ ID NO: 3, 4 or 5 under the control of a promoter which is active in the cell, the transcription of which generates a shRNA molecule.

12. The tumor cell according to claim 9, wherein said molecule of nucleic acid of exogenous origin is introduced into the cell by means of a lentiviral, retroviral, adenoviral or adeno-associated vector or by means of nanoparticles.

13. The tumor cell according to any claim 3, wherein said tumor cell is a primary tumor cell extracted from a patient afflicted with a tumor, modified by adding said agent of exogenous origin, maintained in culture under conditions and for a period which is sufficient to present a negative ERK5 phenoptype and a negative MHC-I phenotype.

14. The tumor cell according to claim 1, wherein said tumor cell is a human cell.

15. The tumor cell according to claim 1, wherein said tumor cell is a lymphocyte, a leukocyte, a breast or prostate cancer cell or a metastatic cell.

16. (canceled)

17. The tumor cell according to claim 1, useful as a medicament or vaccine for the prevention or treatment of a cancer or of the development of metastases in a human or animal patient.

18. The tumor cell according to claim 1, wherein said tumor cell is a primary cell extracted from said patient, modified to present a negative MHC-I phenotype.

19. The tumor cell according to claim 18, further modified to present a negative ERK5 phenotype.

20. The tumor cell according to claim 1, wherein said tumor cell is an allogenic cell deriving from a cell line.

21. The tumor cell according to claim 1, wherein said tumor cell is a tumor cell of the same type as those which are responsible for the cancer with which said patient is afflicted.

22. The tumor cell according to claim 1, wherein said tumor cell is a tumor cell of a type which differs from those which are responsible for the cancer with which said patient is afflicted.

23. The tumor cell according to claim 17, wherein said cancer is a leukemia, a lymphoma, a myeloma, a breast cancer or a prostate cancer.

24. The tumor cell according to claim 23, wherein said tumor cell is a B or T lymphocyte.

25. The tumor cell according to claim 1, wherein said vaccine or medicament is to be administered in combination with an allogenic bone marrow or blood cell transplant.

26. The living tumor cell according to claim 1, wherein said vaccine or medicament is to be administered in combination with another anti-cancer treatment.

27. The living tumor cell according to claim 1, wherein said vaccine or medicament is to be administered in combination with the administration of activated NK cells.

28. The living tumor cell according to claim 1, wherein said vaccine or medicament is to be administered locally in the near vicinity of endogenous tumor cells of the patient, in the near vicinity of or into an organ of said patient afflicted by said cancer, or into the blood system.

29. A tumor cell line obtained from a tumor cell according to claim 3.

30. An in vitro or ex vivo method for modulating the overall level of expression or the overall quantity of MHC-I proteins present at the surface of a living animal tumor cell, comprising
a) culturing an isolated or cultured animal tumor cell
(i) in a medium which favors the fermentation metabolic pathway at the expense of the respiration metabolic pathway, or
(ii) in a medium which favors the respiration metabolic pathway at the expense of the fermentation metabolic pathway;
for a period of time sufficient to allow the overall level of expression or the overall quantity of MHC-I proteins present at the surface of said tumor cell to be modulated, and
b) recovering said tumor cell.

31. (canceled)

32. (canceled)

33. (canceled)

34. (canceled)

35. (canceled)

36. (canceled)

37. (canceled)

38. (canceled)

39. An in vitro or ex vivo method for obtaining an animal tumor cell presenting a negative ERK5 phenotype and a negative MHC-I phenotype, comprising:
a) introducing into an isolated or cultured animal tumor cell a first exogenous agent which is capable under appropriate culture conditions of reducing the level of expression or the quantity of the endogenous ERK5 protein and possibly also the overall level of expression or the overall quantity of MHC-I proteins at the surface of said cell;
b) culturing said tumor cell under conditions and for a period of time which are sufficient for said cell to present a negative ERK5 and negative MHC-I phenotype,
c) recovering said cell.

40. (canceled)

41. (canceled)

42. (canceled)

43. (canceled)

44. An in vitro or ex vivo method for obtaining activated NK cells, comprising:
(i) in vitro or ex viva contacting living NK cells with living tumor cells presenting a negative MHC-I phenotype under conditions and for a duration sufficient to induce activation of the NK cells;
(ii) recovering said activated NK cells.

45. (canceled)

46. (canceled)

47. (canceled)

48. (canceled)

49. (canceled)

50. (canceled)

51. (canceled)

52. (canceled)

53. (canceled)

54. A method for preventing or treating a cancer in a patient comprising administering to the patient an agent which is capable of endowing a human or animal tumor cell with a negative ERK5 phenotype and a negative MHC-I phenotype.

55. (canceled)

56. (canceled)

57. (canceled)

58. (canceled)

59. (canceled)

60. (canceled)

61. A therapeutic composition or vaccine comprising cells according to claim 1 and a pharmaceutically acceptable vehicle.

62. (canceled)

63. (canceled)
